(19) United States
(12) Patent Application Publication
(10) Pub. No.: US 2026/0207527 A1
(43) Pub. Date: Jul. 23, 2026

(54) STABILIZED LIPID NANOPARTICLES FOR THE DELIVERY OF NUCLEIC ACIDS

(71) Applicant: CASE WESTERN RESERVE UNIVERSITY, Cleveland, OH (US)

(72) Inventors: Zheng-Rong Lu, Cleveland, OH (US); Da Sun, Cleveland, OH (US)

(21) Appl. No.: 19/138,882

(22) PCT Filed: Dec. 18, 2023
(86) PCT No.: PCT/US2023/084590

§ 371 Date: Jun. 13, 2025

Related U.S. Application Data
(60) Provisional application No. 63/387,763, filed on Dec. 16, 2022.

(51) Int. Cl. A61K 9/51; A61K 31/7105

(52) U.S. Cl. CPC A61K 9/5123; A61K 31/7105

(57) ABSTRACT

A stabilized lipid nanoparticle includes mRNA and a compound comprising formula (I):

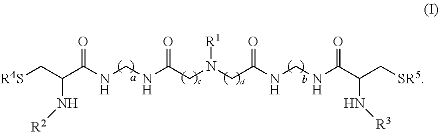

### RELATED APPLICATION

This application claims priority from U.S. Provisional Application No. 63/387,763, filed Dec. 16, 2022, the subject matter of which is incorporated herein by reference in its entirety.

### GOVERNMENT FUNDING

This invention was made with government support under CA235152 awarded by the National Institutes of Health. The government has certain rights in the invention.

### BACKGROUND

Delivery of biologically active substances such as small molecule drugs, proteins, and nucleic acids including mRNA is a medical challenge. In particular, the delivery of nucleic acids to cells is made difficult by the relative instability and low cell permeability of such molecules. Currently approved lipid nanoparticle (LNP) compositions require a mixture of four components: phospholipid(s); cholesterol; PEGylated lipid(s); and cationic or ionizable lipid(s), e.g., for delivery mRNA vaccines. The phospholipids and cholesterol are used to provide the necessary structure and stability, the PEGylated lipids support prolonged circulation, and the cationic/ionizable lipids are for complexing of the negatively charged mRNA molecules and enable the exit of the mRNA from the endosome to the cytosol for translation. There exists a need to develop compositions and methods for improved delivery of therapeutic and/or prophylactics molecules into cells or organs.

### SUMMARY

Embodiments described herein relate to stabilized lipid nanoparticles that include a plurality of pH sensitive protonatable or ionizable lipids. The plurality of pH sensitive protonatable or ionizable lipids can form multifunctional pH-sensitive carriers that are designed to condense nucleic acids, such as mRNA, and deliver the condensed nucleic acids to cells.

In some embodiments, the stabilized lipid nanoparticles can include:
a) a plurality of pH sensitive protonatable or ionizable lipids having the structure of formula (I):

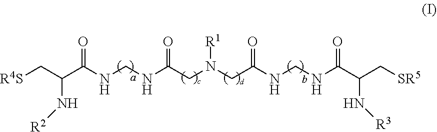

wherein R¹ is an alkyl group or an aromatic group, each of which is optionally substituted one or more hydroxyl group, ether group, or amino group;
R² and R³ are independently an aliphatic group or a hydrophobic group;
R⁴ and R⁵ are independently H, an alkyl group, an alkenyl group, an acyl group, or an aromatic group, or each R⁴ or R⁵ independently includes a polymer, or a polysaccharide, wherein each R⁴ or R⁵ is optionally substituted with a targeting group;
a, b, c, and d are independently an integer from 1 to 10; and pharmaceutically acceptable salts thereof;
b) at least one mRNA complexed with and/or encapsulated by the pH sensitive protonatable or ionizable lipids; and
c) a stabilizing amount of at least one stabilizing polymer, polysaccharide, or structural lipid that is conjugated to and/or complexed with the pH sensitive protonatable or ionizable lipids.

In some embodiments, R¹ comprises at least one of:

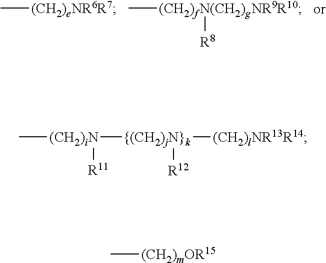

where R⁶, R⁷, R⁸, R⁹, R¹⁰, R¹¹, R¹², R¹³, R¹⁴, and R¹⁵ are independently hydrogen, an alkyl group, a hydrophobic group, a nitrogen containing substituent, or an oxygen containing substituent; and
e, f, g, i, j, k, l, and m are an integer from 1 to 10.

In some embodiments, wherein a, b, c, and d are each 2.

In some embodiments, R¹ comprises at least one of CH₂CH₂NH₂, CH₂CH₂OH, CH₂CH₂OCH₂CH₂OH, CH₂CH₂OCH₂CH₂NH₂, CH₂CH₂NHCH₂CH₂NHCH₂CH₂NH, or CH₂CH₂NHCH₂CH₂CH₂CH₂NHCH₂CH₂CH₂NH.

In some embodiments, R² and R³ are each independently a saturated alkyl with long or branched chains, and or a fatty acid hydrophobic group derived from oleic acid or linoleic acid.

In other embodiments, R² and R³ are the same or different.

In some embodiments, at least one of R⁴ or R⁵ includes a polymer, or a polysaccharide, each optionally substituted with targeting group. The targeting group can be for example, an antibody, antibody fragment, nanobody, peptide saccharide, organic compound, etc.

In some embodiments, the polymer or the polysaccharide of at least one of R⁴ or R⁵ is the stabilizing polymer or the stabilizing polysaccharide.

In some embodiments, the polymer can include polyethylene glycol (PEG). The PEG can have an average molecular weight of about 1,000 Daltons to about 100,000 Daltons, preferably about 1,000 Daltons to about 50,000 Daltons, more preferably about 1,000 Daltons to about 20,000 Daltons, or about 2,000 Daltons to about 10,000 Daltons.

In some embodiments, the polysaccharide can include dextran. The dextran can have an average molecular weight of about 1,000 to about 50,000 Daltons, more preferably about 1,000 Daltons to about 20,000 Daltons, or about 2,000 Daltons to about 10,000 Daltons.

In some embodiments, the dextran includes at one least side chain functionalized with a maleimide linker that is conjugated to a thiol group of formula (I) and/or a terminal end functionalized with a maleimide linker that is conjugated to a thiol group of formula (I).

In some embodiments, for at least some of the pH sensitive protonatable or ionizable lipids, R⁴ and R⁵ are each H, and for other of the pH sensitive protonatable or ionizable lipids, at least one of R⁴ or R⁵ includes a polymer, or a polysaccharide, each optionally substituted with targeting group.

In some embodiments, the pH sensitive protonatable or ionizable lipids are selected from:

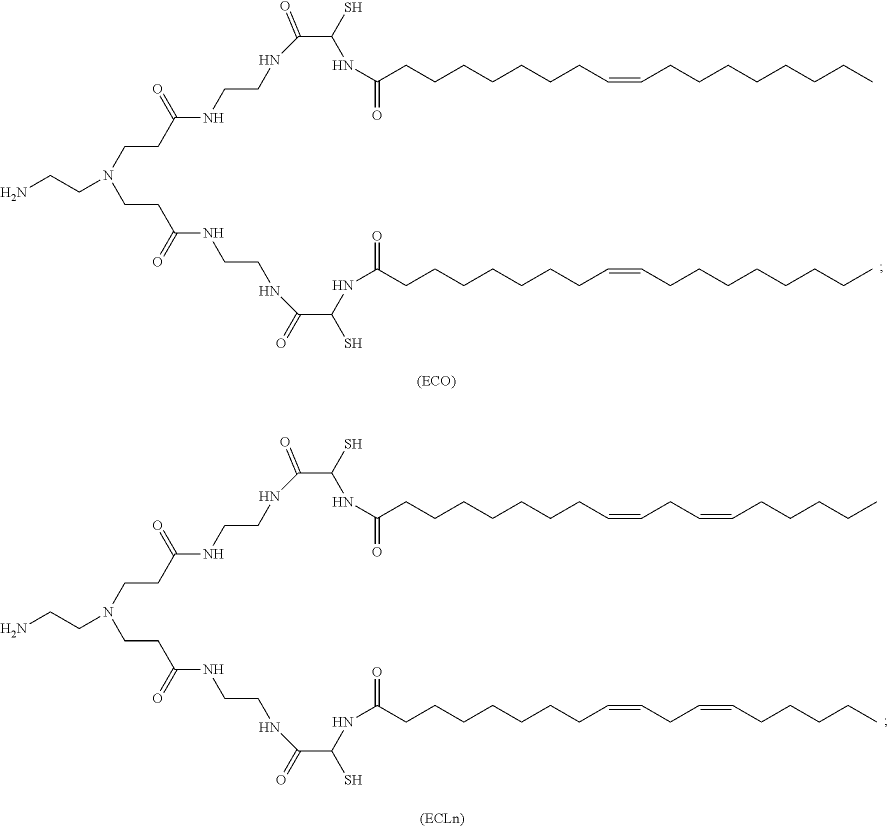 [SMILES: CCCCCCCC/C=C\CCCCCCCC(=O)NC(S)C(=O)NCCNC(=O)CCN(CCN)CCC(=O)NCCNC(=O)C(S)NC(=O)CCCCCCC/C=C\CCCCCCCC ; CCCCC/C=C\C/C=C\CCCCCCCC(=O)NC(S)C(=O)NCCNC(=O)CCN(CCN)CCC(=O)NCCNC(=O)C(S)NC(=O)CCCCCCC/C=C\C/C=C\CCCCC]

polyethylene glycol (PEG) modified lipids thereof, dextran modified lipids thereof, or combinations thereof.

In some embodiments, the plurality of pH sensitive protonatable or ionizable lipids include a plurality of ECO and/or ECLn lipids and a plurality of ECLn and/or ECO modified with PEG and/or dextran.

In some embodiments, the plurality of pH sensitive protonatable or ionizable lipids include about 1 mol % to about 30 mol %, about 1 mol % to about 25 mol %, or about 1 mol % to about 20 mol %, about 1 mol % to about 15 mol %, or about 2.5 mol % to about 15 mol % of ECLn and/or ECO modified with PEG and/or dextran.

In some embodiments, the targeting group is covalently attached to the polymer or polysaccharide.

In some embodiments, the stabilizing polysaccharide is hyaluronic acid that is complexed with the plurality of pH sensitive protonatable or ionizable lipids and mRNA. The hyaluronic acid can provide a surface modification of the stabilized lipid nanoparticle and have an average molecular weight of about 1,000 Daltons to about 100,000 Daltons, preferably about 1,000 Daltons to about 50,000 Daltons, more preferably about 1,000 Daltons to about 20,000 Daltons, or about 2,000 Daltons to about 10,000 Daltons. The stabilized lipid nanoparticle can include, for example, about 1 mol % to about 30 mol %, about 1 mol % to about 25 mol %, or about 1 mol % to about 20 mol %, about 1 mol % to about 15 mol %, about 2.5 mol % to about 15 mol %, about 2.5 mol % to about 10 mol %, or about 2.5 mol % to about 5 mol % hyaluronic acid.

In some embodiments, the structural lipid can include cholesterol. For example, the stabilized lipid nanoparticle can include about 1 mol % to about 30 mol %, about 1 mol % to about 25 mol %, or about 1 mol % to about 20 mol %, about 1 mol % to about 15 mol %, about 2.5 mol % to about 15 mol %, about 2.5 mol % to about 10 mol %, or about 2.5 mol % to about 5 mol % cholesterol.

In some embodiment, the stabilized lipid nanoparticle can have an N/P ratio of about 2 to about 20, about 2 to about 18, about 2 to about 16, about 2 to about 14, about 2 to about 12, or about 4 to about 12.

In some embodiments, the stabilized lipid nanoparticle can have an average particle diameter of about 100 nm to less than about 500 nm, about 100 nm to about 400 nm, about 100 nm to about 300 nm, or about 100 nm to about 200 nm.

Other embodiments described herein relate to a pharmaceutical composition that includes a plurality of stabilized lipid nanoparticles as described herein.

In some embodiment, the pharmaceutical composition can include at least one excipient. The excipient can include a cryoprotectant, such as sucrose, that is provided in the pharmaceutical composition at an amount effective to enhance the stability of the composition when cryopreserved.

Other embodiments describe herein relate to a vaccine that includes a plurality of stabilized lipid nanoparticles as described herein or the pharmaceutical composition described herein.

### BRIEF DESCRIPTION OF THE DRAWINGS

FIGS. 1(A-B) is a characterization of ECO/mRNA nanoparticles formulated at different N/P ratios (N/P=4,6,8,10,12). DLS of size distribution, size, zeta potential and polydispersity index (PDI) (A), and mRNA encapsulation (B) by agarose gel electrophoresis.

FIGS. 2(A-C) illustrate GFP expression in ARPE-19 cells after transfection of ECO/mRNA nanoparticles of different N/P ratios. Confocal microscopic images (A), fluorescent intensities (B) and cell viability of GFP expressions 24 and 48 h after transfections.

FIGS. 3(A-B) illustrate GFP expression in ARPE-19 cells after transfection of ECO/mRNA nanoparticles of different mRNA doses. Confocal microscopic images (A), and fluorescent intensities (B) of GFP expressions 24 and 48 h after transfections.

FIG. 4 GFP expression in HEK293T cells after transfection of ECO/mRNA nanoparticles. Confocal microscopic images of GFP expressions 24, 48 and 72 h after transfections with Lipofectamine as a control.

FIGS. 5(A-B) illustrate Characterization of PEGylated ECO/mRNA nanoparticles formulated at different N/P ratios (N/P=4,6,8,10,12). DLS of size distribution, size, zeta potential and polydispersity index (PDI) (A), and mRNA encapsulation (B) by agarose gel electrophoresis.

FIGS. 6(A-C) illustrate GFP expression in ARPE-19 cells after transfection of PEGylated ECO/mRNA nanoparticles of different N/P ratios. Confocal microscopic images (A), fluorescent intensities (B) and cell viability of GFP expressions 24 and 48 h after transfections.

FIGS. 7(A-B) illustrate GFP expression in ARPE-19 cells after transfection of PEGylated ECO/mRNA nanoparticles of different mRNA doses. Confocal microscopic images (A), and fluorescent intensities (B) of GFP expressions 24 and 48 h after transfections.

FIGS. 8(A-B) illustrate Stability of PEGylated ECO/mRNA nanoparticles under PBS incubation and after freeze thaw cycles. DLS of sizes, size distributions and PDIs after PBS incubation (A) and freeze-thaw cycles (B).

FIG. 9 illustrates GFP expression in the nude mouse 6 h after receiving muscle injection of PEGylated RGD-PEG-ECO/mGFP nanoparticles indicated by green, fluorescent images. The left leg was injected with freshly made nanoparticles, while the right leg of the same mouse was injected with the same nanoparticles 1 week after storage under −80° C.

FIG. 10 illustrates GFP fluorescent intensity subtracted from the non-treated control in the nude mouse 6 h after receiving muscle injection of PEGylated ECO/mGFP nanoparticles. For the nude mouse, the left leg was injected with freshly made nanoparticles, while the right leg of the same mouse was injected with the same nanoparticles 1 week after storage under −80° C.

FIG. 11 illustrates bioluminescence of luciferase expression in the nude mice 6 h after receiving muscle injection of PEGylated ECO/mLuc nanoparticles in the right leg. (1) and (2) are two replicates.

FIG. 12 illustrates quantitative analysis of bioluminescence of luciferase expression in the nude mice 6 h after receiving muscle injection of PEGylated ECO/mLuc nanoparticles in the right leg. (1) and (2) are two replicates.

FIG. 13 illustrates hyaluronic acid modified ECO/mRNA nanoparticle (N/P=8) formulation. DLS of size and zeta potential distributions.

FIG. 14 illustrates agarose gel electrophoresis evaluating the encapsulation and stability of ECO/mRNA nanoparticle formulations with hyaluronic acid (HA).

FIG. 15 illustrates GFP expression in ARPE-19 cells after transfection of HA modified ECO/mRNA nanoparticles. Confocal microscopic images of GFP expressions 48 h after transfections. HA was added to the ECO/mRNA nanoparticles as surface modification at both 2.5 and 5 mol %.

FIG. 16 illustrates GFP expression in ARPE-19 cells after transfection of HA modified ECO/mRNA nanoparticles. Confocal microscopic images of GFP expressions 48 h after transfections. HA was added to the ECO/mRNA nanoparticles as surface modification at both 2.5 and 5 mol %.

FIG. 17 illustrates ECO/mRNA nanoparticles formulated with cholesterol (Chol) (N/P=8). DLS of size distributions of ECO/mRNA nanoparticles formulated with 0, 1, 2, 5, and 10 mol % of Chol.

FIG. 18 illustrates ECO/mRNA nanoparticles formulated with cholesterol (Chol) (N/P=8). DLS of zeta potential distributions of ECO/mRNA nanoparticles formulated with 0, 1, 2, 5, and 10 mol % of Chol.

FIG. 19 illustrates agarose gel electrophoresis evaluating the encapsulation and stability of ECO/mRNA nanoparticles formulated with 0, 1, 2, 5, and 10 mol % of Chol.

FIG. 20 illustrates GFP expression in HEK293T cells after transfection of ECO/mRNA nanoparticles formulated with cholesterol (1, 2, 5, and 10 mol % regarding the amount of ECO). Confocal microscopic images of GFP expressions 24 and 48 h after transfections.

FIG. 21 illustrates Size distributions of ECO/mRNA nanoparticles formulated with cholesterol (Chol) (N/P=8) after 2 weeks storage under −20° C. DLS of size distributions of ECO/mRNA nanoparticles formulated with 0, 1, 2, 5, and 10 mol % of Chol after 2 weeks storage under −20° C. compared with freshly made nanoparticles.

FIG. 22 illustrates zeta potential distributions of ECO/mRNA nanoparticles formulated with cholesterol (Chol) (N/P=8) after 2 weeks storage under −20° C. DLS of zeta potential distributions of ECO/mRNA nanoparticles formulated with 0, 1, 2, 5, and 10 mol % of Chol after 2 weeks storage under −20° C.

FIG. 23 illustrates agarose gel electrophoresis evaluating the encapsulation and stability of ECO/mRNA nanoparticles formulated with 0, 1, 2, 5, and 10 mol % of Chol after 2 weeks storage under −20° C.

FIG. 24 illustrates GFP expression in HEK293T cells after transfection of ECO/mRNA nanoparticles formulated with cholesterol (1, 2, 5, and 10 mol % regarding the amount of ECO) after 2 weeks storage under −20° C. Confocal microscopic images of GFP expressions 24, 48 and 72 h after transfections.

FIG. 25 illustrates the preparation of end functionalized dextran-mono-mal (10 kDa).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like. “Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

The terms “approximately” and “about,” as applied to one or more values of interest, refer to a value that is similar to a stated reference value. In certain embodiments, the term “approximately” or “about” may refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “alkenyl group” is defined herein as a C₂-C₂₀ alkyl group possessing at least one C═C double bond.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “acyl” group as used herein is represented by the formula C(O)R, where R is an organic group such as, for example, an alkyl or aromatic group as defined herein.

The term “alkylene group” as used herein is a group having two or more CH₂ groups linked to one another. The alkylene group can be represented by the formula (CH₂)ₐ, where a is an integer of from 2 to 25.

The term “aromatic group” as used herein is any group containing an aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

The phrase “nitrogen containing substituent” is defined herein as any amino group. The term “amino group” is defined herein as a primary, secondary, or tertiary amino group. In the alternative, the nitrogen containing substituent can be a quaternary ammonium group. The nitrogen containing substituent can be an aromatic or cycloaliphatic group, where the nitrogen atom is either part of the ring or directly or indirectly attached by one or more atoms (i.e., pendant) to the ring. The nitrogen containing substituent can be an alkylamino group having the formula RNH₂, where R is a branched or straight alkyl group, and the amino group can be substituted or unsubstituted.

The term “encapsulation,” or its grammatical equivalent, refers to the process of confining a nucleic acid molecule within a nanoparticle.

The term “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and their grammatical equivalents, are used interchangeably.

The terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein.

The term “impurities” refers to substances inside a confined amount of liquid, gas, or solid, which differ from the chemical composition of the target material or compound. Impurities are also referred to as contaminants.

The term “in vitro” refers to events that occur in an artificial environment, e.g., in a test tube or reaction vessel, in cell culture, etc., rather than within a multi-cellular organism.

The term “in vivo” refers to events that occur within a multi-cellular organism, such as a human and a non-human animal. In the context of cell-based systems, the term may be used to refer to events that occur within a living cell (as opposed to, for example, in vitro systems).

The term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

The terms “local distribution,” “local delivery,” or grammatical equivalent, refer to tissue specific delivery or distribution. Typically, local distribution or delivery requires a peptide or protein (e.g., enzyme) encoded by mRNAs be translated and expressed intracellularly or with limited secretion that avoids entering the patient's circulation system.

The term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “mRNA integrity” refers to the quality of mRNA. In particular, mRNA integrity refers to the percentage of mRNA that is not degraded. mRNA integrity may be determined using methods well known in the art, for example, by RNA agarose gel electrophoresis (e.g., Ausubel et al., John Weley & Sons, Inc., 1997, Current Protocols in Molecular Biology) or capillary electrophoresis. In some embodiments, mRNA integrity can be quantified and expressed as a percent. For example, capillary electrophoresis and similar methods can be uses to separate degraded mRNA from mRNA that is not degraded and then the percent integrity, i.e., percent of mRNA not degraded relative to total mRNA, can be calculated based on the relative areas from the resulting chromatogram.

The term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle.

The term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, sulfonate and aryl sulfonate. Further pharmaceutically acceptable salts include salts formed from the quaternization of an amine using an appropriate electrophile, e.g., an alkyl halide, to form a quarternized alkylated amino salt.

The term “potency,” or grammatical equivalents, refers to level of expression of protein(s) or peptide(s) that the mRNA encodes and/or the resulting biological effect.

The term “salt” refers to an ionic compound that does or may result from a neutralization reaction between an acid and a base.

The terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

The term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “substantially free” refers to a state in which relatively little or no amount of a substance to be removed (e.g., prematurely aborted RNA sequences) are present. For example, “substantially free of prematurely aborted RNA sequences” means the prematurely aborted RNA sequences are present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) of the impurity. Alternatively, “substantially free of prematurely aborted RNA sequences” means the prematurely aborted RNA sequences are present at a level less than about 100 ng, 90 ng, 80 ng, 70 ng, 60 ng, 50 ng, 40 ng, 30 ng, 20 ng, 10 ng, 1 ng, 500 μg, 100 pg, 50 μg, 10 pg, or less.

The term “target tissues” refers to any tissue that is affected by a disease to be treated. In some embodiments, target tissues include those tissues that display disease-associated pathology, symptom, or feature.

The term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin D, vitamin E (alpha-tocopherol), vitamin C, vitamin K, xylitol, and other species disclosed herein.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

A “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell. In certain aspects, the stabilized lipid nanoparticles described herein are free of phospholipids, i.e., does not have the phospholipid component used in the traditional lipid nanoparticle compositions.

The term “polypeptide” or “polypeptide of interest” refers to a polymer of amino acid residues typically joined by peptide bonds that can be produced naturally (e.g., isolated or purified) or synthetically.

As used herein, “size” or “mean size” in the context of nanoparticle compositions refers to the mean diameter of a nanoparticle composition.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

Embodiments described herein relate to stabilized lipid nanoparticles that include a plurality of pH sensitive protonatable or ionizable lipids. The plurality of pH sensitive protonatable or ionizable lipids can form multifunctional pH-sensitive carriers that are designed to condense nucleic acids, such as mRNA, and deliver the condensed nucleic acids to cells. Messenger RNAs, which are single stranded, are more structurally liable and unstable than the double stranded DNA or siRNA. It was also estimated that the half-life of the naked double-stranded DNA in the cytosol of mammalian cells is between about 50 and 90 minutes, while the half-life of naked mRNA is reported to be only seconds or less than a second; based on the half-life, mRNA is about 5,500 times more unstable than DNA.

Lipid nanoparticles encapsulating mRNA have to withstand various forces including temperature and pressure changes that can alter the size, PDI, and encapsulation efficiency of mRNA-LNPs while also preserving mRNA integrity. Therefore, maintaining quality and integrity of mRNA encapsulated in lipid nanoparticles during storage and delivery while also maintaining size, PDI, and encapsulation efficiency of mRNA-LNPs is a challenge.

In some embodiments, the pH sensitive protonatable or ionizable lipids of the stabilized lipid nanoparticle can include a protonable amino head group, which can complex with the nucleic acids, such as mRNA, fatty acid or lipid tails, which can participate in hydrophobic condensation, two cysteine residues capable of forming disulfide bridges via autooxidation, and optionally, targeting group that targets and/or binds to protein or other biological target molecule in a subject.

The protonable amino head group can complex with nucleic acids to form stabilized lipid nanoparticles for delivery of nucleic acids to cells. The amines in the head groups contribute to the essential pH-sensitive characteristic of the carrier system, which is important for improving endosomal escape and mRNA expression efficiency. Greater protonation of the amino head groups can occur in the relatively acidic environment (pH=5-6) of the endosome and lysosome compartments after cellular uptake. This enhances electrostatic interactions between the cationic carriers and the anionic phospholipids of endosomal/lysosomal membranes, promoting the bilayer destabilization and nanoparticle charge neutralization events required for efficient cytosolic release of their nucleic acid payload. By affecting the number of amines, and thus overall pKa, of the cationic carrier, the choice of head group can ultimately determine the degree to which such protonation can occur. The pH-sensitive property of the lipid nanoparticles is essential so that the nanoparticles do not affect the integrity of the outer cell membrane and cause cell death, but instead are able to selectively fuse with and destabilize the endosomal and lysosomal membranes.

The cysteine residues can form disulfide bridges via autooxidation and react with functional groups of other compounds, such as those containing thiol groups. Once the nucleic acid is complexed with the compound, the thiol groups can produce disulfide (S—S) bonds or bridges by autooxidation to form oligomers and polymers or cross-linking. The disulfide bonds can stabilize the lipid nanoparticles and help achieve release of the nucleic acid once the nanoparticle is in the cell.

For example, when the nucleic acid is mRNA, the cleavage of disulfide bonds in the mRNA delivery system in reductive cytoplasm can facilitate cytoplasm-specific release of mRNA. The lipid nanoparticles can be stable in the plasma at very low free thiol concentration (e.g., 15 μM). When the lipid nanoparticles are incorporated into target cells, the high concentration of thiols present in the cell (e.g., cytoplasm) will reduce the disulfide bonds to facilitate the dissociation and release of the mRNA.

The fatty acid or lipid tails groups can participate in hydrophobic condensation and help form compact, stable nanoparticles with the nucleic acids and introduce amphiphilic properties to facilitate pH sensitive escape of nanoparticles from endosomal and lysosomal compartments. This is particularly useful when the nanoparticles are used as in vivo delivery devices.

In general, the transfection efficiency of lipid nanoparticles has been shown to decrease with increasing alkyl chain length and saturation of the lipid tail groups. When saturated, shorter aliphatic chains (C12 and C14) favor higher rates of inter-membrane lipid mixing and reportedly allow for better transfection efficiencies in vitro, as compared to in vivo, whereas the opposite is true for longer chains (C16 and C18). Typically, saturated fatty acids greater than 14 carbons in length are not favorable for nucleic acid transfections due to their elevated phase transition temperature and overall less fluidity than those that are unsaturated. However, it has been discovered that there exists a limit, at which point an increase in unsaturation and lipid fluidity is inversely correlated to transfection efficiency, primarily because some degree of rigidity is required for particle stability, as evidenced by the widespread use of cholesterol in lipid nanoparticle formulations.

Advantageously, the stabilized lipid nanoparticles formed using the plurality of pH sensitive protonatable or ionizable lipids have improved stability when administered systemically to a subject, protect condensed nucleic acids from degradation, and promote endosomal escape and cytosolic release upon cellular uptake.

In some embodiments, a targeting group can be attached to the plurality of pH sensitive protonatable or ionizable lipids by, for example, a thiol group of a cysteine residue. The targeting group can be useful in the delivery of nucleic acids into cells. The targeting group can be a peptide, an antibody, an antibody fragment or one of their derivatives. For example, target-specific peptides can be conjugated directly to the compound or indirectly via a linker (e.g., polyethylene glycol) prior or during the formation of nanoparticles. Depending upon the selection of the targeting group, the targeting group can be covalently bonded to either thiol group of the cysteine residues.

In one aspect, the targeting group is indirectly attached to the plurality of pH sensitive protonatable or ionizable lipids by a linker. Examples of linkers include, but are not limited to, a polyamine group, a polyalkylene group, a polyamino acid group, a polyethylene glycol group, or a polysaccharide group. The selection of the linker as well as the molecular weight of the linker can vary depending upon the desired properties. In one aspect, the linker is polyethylene glycol having a molecular weight from 500 to 10,000, 500 to 9,000, 500 to 8,000, 500 to 7,000, or 2,000 to 5,000. In certain aspects, the targeting group is first reacted with the linker in a manner such that the targeting group is covalently attached to the linker. For example, the linker can possess one or more groups that can react with an amino group present on a peptide. The linker also possesses additional groups that react with and form covalent bonds with the compounds described herein. For example, the linker can possess maleimide groups that readily react with the thiol groups. The selection of functional groups present on the linker can vary depending upon the functional groups present on the compound. In one aspect, the targeting group is a peptide such as, an RGD peptide or bombesin peptide that is covalently attached to polyethylene glycol.

In some embodiments, the linker can include an acid labile bond, such as formed by incorporation of a hydrazone into the linker, that is hydrolysable in an endolysomal environment following uptake to cells, such as cancer cells. For example, the linker can be covalently linked to the pH sensitive protonatable or ionizable lipid of the lipid nanoparticle by at least one of a covalent hydrolysable ester, covalent hydrolysable amide, covalent photodegradable urethane, or covalent hydrolysable acrylate-thiol linkage. Following cellular uptake of the lipid nanoparticle, within the endosomes, the increasingly acidic environment can cleave the acid labile linkage to promote shedding of a polymer linker, such as PEG, and expose the core of the compound/nucleic complex nanoparticle.

In other aspects, it is also desirable to attach the targeting group to a nanoparticle produced by the plurality of pH sensitive protonatable or ionizable lipids described herein. For example, after a nanoparticle composed of a nucleic acid has been produced using the plurality of pH sensitive protonatable or ionizable lipids and techniques described herein, the targeting group can be attached to the nanoparticle via a linker.

In some embodiments, the stabilized nanoparticle construct can include:
a) a plurality of pH sensitive protonatable or ionizable lipids having the structure of formula (I):
the compound can include formula (I):

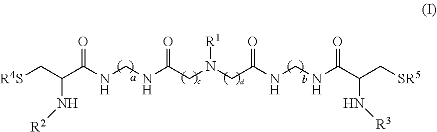

wherein R¹ is an alkyl group or an aromatic group, each of which is optionally substituted one or more hydroxyl group, ether group, or amino group;
R² and R³ are independently an aliphatic group or a hydrophobic group;
R⁴ and R⁵ are independently H, an alkyl group, an alkenyl group, an acyl group, or an aromatic group, or each R⁴ or R⁵ independently includes a polymer, or a polysaccharide, wherein each R⁴ or R⁵ is optionally substituted with a targeting group;
a, b, c, and d are independently an integer from 1 to 10; and pharmaceutically acceptable salts thereof;
b) at least one mRNA complexed with and/or encapsulated by the pH sensitive protonatable or ionizable lipids; and
c) a stabilizing amount of at least one stabilizing polymer, polysaccharide, or structural lipid that is conjugated to and/or complexed with the pH sensitive protonatable or ionizable lipids.

In some embodiments, R¹ can include at least one of:

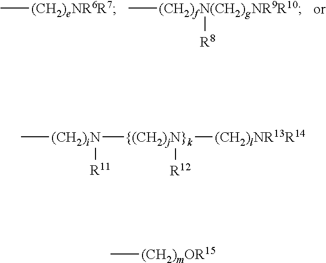

where R⁶, R⁷, R⁸, R⁹, R¹⁰, R¹¹, R¹², R¹³, R¹⁴, and R¹⁵ are independently hydrogen, an alkyl group, a hydrophobic group, a nitrogen containing substituent, or an oxygen containing substituent; and
e, f, g, i, j, k, 1, and m are an integer from 1 to 10.

For example, R¹ can include at least one of CH₂NH₂, CH₂CH₂NH₂, CH₂CH₂CH₂NH₂, CH₂CH₂OH, CH₂CH₂OCH₂CH₂OH, CH₂CH₂OCH₂CH₂NH₂, CH₂CH₂CH₂CH₂NH₂, CH₂CH₂CH₂CH₂CH₂NH₂, CH₂NHCH₂CH₂CH₂NH₂, CH₂CH₂NHCH₂CH₂CH₂NH₂, CH₂CH₂CH₂NHCH₂CH₂CH₂CH₂NHCH₂CH₂CH₂NH₂, CH₂CH₂NHCH₂CH₂CH₂CH₂NH₂, CH₂CH₂NHCH₂CH₂CH₂NHCH₂CH₂CH₂HN₂, or CH₂CH₂NH(CH₂CH₂NH)_{d}CH₂CH₂NH₂, where d is from 0 to 10.

In some embodiments, R¹ can be CH₂CH₂NH₂ or CH₂CH₂NHCH₂CH₂CH₂NHCH₂CH₂CH₂HN₂. In other embodiments, R¹ is CH₂CH₂NH₂.

In other embodiments, R² and R³ are independently an aliphatic group or a hydrophobic group derived from fatty acid, such as oleic acid or linoleic acid, and are the same or different. The additional double bond in linoleic acid introduces an extra kink into the hydrocarbon backbone, giving the compound a broader conical shape than oleic acid and increasing its fluidity. When incorporated into a nanoparticle structure, the extra degree of unsaturation elevates the propensity to form the hexagonal phase during an impending membrane fusion event of cellular uptake.

In some embodiments, R⁴ and R⁵ are independently H, an alkyl group, an alkenyl group, an acyl group, or an aromatic group, or each R⁴ or R⁵ independently includes a polymer, or a polysaccharide, wherein each R⁴ or R⁵ is optionally substituted with targeting group.

In some embodiments, R² and R³ are each independently a saturated alkyl with long or branched chains, and or a fatty acid hydrophobic group derived from oleic acid or linoleic acid.

In other embodiments, R² and R³ are the same or different.

In some embodiments, at least one of R⁴ or R⁵ includes a polymer, or a polysaccharide, each optionally substituted with targeting group. The targeting group can be for example, an antibody, antibody fragment, nanobody, peptide saccharide, organic compound, etc.

In some embodiments, the polymer or the polysaccharide of at least one of R⁴ or R⁵ is the stabilizing polymer or the stabilizing polysaccharide.

In some embodiments, for at least some of the pH sensitive protonatable or ionizable lipids R⁴ and R⁵ are each H and for other of the pH sensitive protonatable or ionizable lipids at least one of R⁴ or R⁵ includes a polymer, or a polysaccharide, each optionally substituted with targeting group.

The surface of the lipid nanoparticles can be modified by, for example, covalently incorporating a polymer, such as polyethylene glycol, by reacting unpolymerized free thiol of the nanoparticle to reduce non-specific tissue uptake in vivo. For example, PEG-maleimide reacts rapidly with free thiol groups. The molecular weight of the PEG can vary depending upon the desired amount of hydrophilicity to be imparted on the carrier. PEG-modification of the carrier can also protect nanoparticles composed of the nucleic acid from enzymatic degradation upon uptake by the cell (e.g., endonucleases). Targeting groups, including peptides, proteins, antibodies or antibody fragment, can also be incorporated into the nanoparticle complexes during the preparation of the complexes to enhance the delivery specificity and efficiency of the genetic materials to the target cells. Polyethylene glycol can be used as the spacer to conjugate targeting agents to the nanoparticle complexes.

In some embodiments, the polymer or stabilizing polymer can include polyethylene glycol (PEG). The PEG can have an average molecular weight of about 1,000 Daltons to about 100,000 Daltons, preferably about 1,000 Daltons to about 50,000 Daltons, more preferably about 1,000 Daltons to about 20,000 Daltons, or about 2,000 Daltons to about 10,000 Daltons.

In some embodiments, the polysaccharide or stabilizing polysaccharide can include dextran. The dextran can have an average molecular weight of about 1,000 to about 50,000 Daltons, more preferably about 1,000 Daltons to about 20,000 Daltons, or about 2,000 Daltons to about 10,000 Daltons.

In some embodiments, the dextran includes at one least side chain functionalized with a maleimide linker that is conjugated to a thiol group of formula (I) and/or a terminal end functionalized with a maleimide linker that is conjugated to a thiol group of formula (I).

In some embodiments, the targeting group is covalently attached to the polymer or polysaccharide.

In some embodiments, the pH sensitive protonatable or ionizable lipids are selected from:

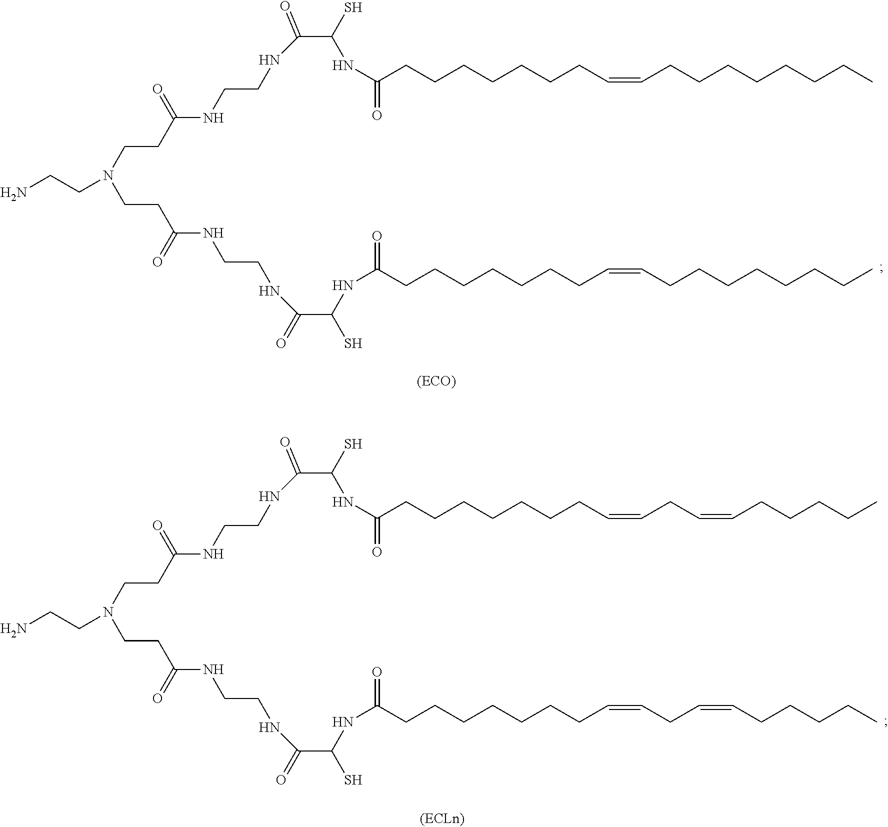 [SMILES: CCCCCCCC/C=C\CCCCCCCC(=O)NC(S)C(=O)NCCNC(=O)CCN(CCN)CCC(=O)NCCNC(=O)C(S)NC(=O)CCCCCCC/C=C\CCCCCCCC ; CCCCC/C=C\C/C=C\CCCCCCCC(=O)NC(S)C(=O)NCCNC(=O)CCN(CCN)CCC(=O)NCCNC(=O)C(S)NC(=O)CCCCCCC/C=C\C/C=C\CCCCC]

polyethylene glycol (PEG) modified lipids thereof, dextran modified lipids thereof, or combinations thereof.

In some embodiments, the plurality of pH sensitive protonatable or ionizable lipids include a plurality of ECO and/or ECLn lipids and a plurality of ECLn and/or ECO modified with PEG and/or dextran.

In some embodiments, the plurality of pH sensitive protonatable or ionizable lipids include about 1 mol % to about 30 mol %, about 1 mol % to about 25 mol %, or about 1 mol % to about 20 mol %, about 1 mol % to about 15 mol %, or about 2.5 mol % to about 15 mol % of ECLn and/or ECO modified with PEG and/or dextran.

In other embodiments, the stabilizing polysaccharide is hyaluronic acid that is complexed with the plurality of pH sensitive protonatable or ionizable lipids and mRNA. The hyaluronic acid can provide a surface modification of the lipid nanoparticle and have an average molecular weight of about 1,000 Daltons to about 100,000 Daltons, preferably about 1,000 Daltons to about 50,000 Daltons, more preferably about 1,000 Daltons to about 20,000 Daltons, or about 2,000 Daltons to about 10,000 Daltons. The stabilized lipid nanoparticle can include, for example, about 1 mol % to about 30 mol %, about 1 mol % to about 25 mol %, or about 1 mol % to about 20 mol %, about 1 mol % to about 15 mol %, about 2.5 mol % to about 15 mol %, about 2.5 mol % to about 10 mol %, or about 2.5 mol % to about 5 mol % hyaluronic acid.

In some embodiments, the lipid nanoparticle can further include one or more structural lipids. Structural lipids can be selected from cholesterol, fecosterol, sitosterol, ergosterol, campesterol, stigmasterol, brassicasterol, tomatidine, tomatine, ursolic acid, alpha-tocopherol, or a mixture thereof. In some embodiments, the structural lipid is cholesterol.

In some embodiments, the structural lipid can include cholesterol. For example, the stabilized lipid nanoparticle can include about 1 mol % to about 30 mol %, about 1 mol % to about 25 mol %, or about 1 mol % to about 20 mol %, about 1 mol % to about 15 mol %, about 2.5 mol % to about 15 mol %, about 2.5 mol % to about 10 mol %, or about 2.5 mol % to about 5 mol % cholesterol.

The pH sensitive protonatable or ionizable lipids having the general formula I can be synthesized using solid phase techniques known in the art. In general, the approach involves the systematic protection/elongation/deprotection to produce a dithiol compound. The hydrophobic group is produced by reacting oleic acid with the amino group present on the cysteine residue.

Any of the pH sensitive protonatable or ionizable lipids described herein can exist or be converted to the salt thereof. In one aspect, the salt is a pharmaceutically acceptable salt. The salts can be prepared by treating the free acid with an appropriate amount of a chemically or pharmaceutically acceptable base. Representative chemically or pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C., such as at room temperature. The molar ratio of the compound to base used is chosen to provide the ratio desired for any particular salts. For preparing, for example, the ammonium salts of the free acid starting material, the starting material can be treated with approximately one equivalent of base to yield a salt.

If the pH sensitive protonatable or ionizable lipids possess carboxylic acid groups, these groups can be converted to pharmaceutically acceptable esters or amides using techniques known in the art. Alternatively, if an ester is present on the dendrimer, the ester can be converted to a pharmaceutically acceptable ester using transesterification techniques.

The pH sensitive protonatable or ionizable lipids described herein have numerous applications with respect to the delivery of nucleic acids to a subject. In some embodiments, the stabilized lipid nanoparticles described herein can be used in gene therapy to deliver nucleic acid or genetic materials to cells and tissues.

In certain embodiments, the mRNA can be a therapeutic and/or prophylactic mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. While exemplary polypeptides include polypeptides from respiratory syncytial virus (RSV) and Covid-19, the pH sensitive protonatable or ionizable lipids and lipid nanoparticles are applicable to any mRNA molecules encoding any polypeptides of interest.

mRNAs may be synthesized according to any of a variety of known methods. Various methods are described in published U.S. Application No. US 2018/0258423, and can be used to practice the present invention, all of which are incorporated herein by reference. For example, mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, the mRNA has an mRNA sequence encoding a protein or a peptide. In some embodiments, the mRNA sequence is codon optimized for efficient expression human cells. In some embodiments, the mRNA sequence is naturally-occurring or a wild-type sequence. In some embodiments, the mRNA sequence encodes a protein or a peptide that contains one or mutations in amino acid sequence.

In some embodiments, the mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO 2011/012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US 2012/0195936 and international publication WO 2011/012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

In some embodiments, mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized as described herein may be used without further purification. In some embodiments, mRNA synthesized may be further purified. Various methods may be used to purify mRNA synthesized as described herein. For example, purification of mRNA can be performed using centrifugation, filtration and for chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein.

The mRNA composition described herein is substantially free of contaminants comprising short abortive RNA species, long abortive RNA species, double-stranded RNA (dsRNA), residual plasmid DNA, residual in vitro transcription enzymes, residual solvent and/or residual salt.

The mRNA composition described herein can have a purity of about between 60% and about 100%. Accordingly, in some embodiments, the purified mRNA has a purity of about 60%. In some embodiments, the purified mRNA has a purity of about 65%. In some embodiments, the purified mRNA has a purity of about 70%. In some embodiments, the purified mRNA has a purity of about 75%. In some embodiments, the purified mRNA has a purity of about 80%. In some embodiments, the purified mRNA has a purity of about 85%. In some embodiments, the purified mRNA has a purity of about 90%. In some embodiments, the purified mRNA has a purity of about 91%. In some embodiments, the purified mRNA has a purity of about 92%. In some embodiments, the purified mRNA has a purity of about 93%. In some embodiments, the purified mRNA has a purity of about 94%. In some embodiments, the purified mRNA has a purity of about 95%. In some embodiments, the purified mRNA has a purity of about 96%. In some embodiments, the purified mRNA has a purity of about 97%. In some embodiments, the purified mRNA has a purity of about 98%. In some embodiments, the purified mRNA has a purity of about 99%. In some embodiments, the purified mRNA has a purity of about 100%.

In some embodiments, the mRNA composition described herein has less than 10%, less than 9%, less than 8%, less than 7%, less than 6%, less than 5%, less than 4%, less than 3%, less than 2%, less than 1%, less than 0.5%, and/or less than 0.1% impurities other than full-length mRNA. The impurities include IVT contaminants, e.g., proteins, enzymes, DNA templates, free nucleotides, residual solvent, residual salt, double-stranded RNA (dsRNA), prematurely aborted RNA sequences (“shortmers” or “short abortive RNA species”), and/or long abortive RNA species. In some embodiments, the purified mRNA is substantially free of process enzymes.

In some embodiments, residual plasmid DNA in the purified mRNA described herein is less than about 1 pg/mg, less than about 2 pg/mg, less than about 3 pg/mg, less than about 4 pg/mg, less than about 5 pg/mg, less than about 6 pg/mg, less than about 7 pg/mg, less than about 8 pg/mg, less than about 9 pg/mg, less than about 10 pg/mg, less than about 11 pg/mg, or less than about 12 pg/mg. Accordingly, the residual plasmid DNA in the purified mRNA is less than about 1 pg/mg. In some embodiments, the residual plasmid DNA in the purified mRNA is less than about 2 pg/mg. In some embodiments, the residual plasmid DNA in the purified mRNA is less than about 3 pg/mg. In some embodiments, the residual plasmid DNA in the purified mRNA is less than about 4 pg/mg. In some embodiments, the residual plasmid DNA in the purified mRNA is less than about 5 pg/mg. In some embodiments, the residual plasmid DNA in the purified mRNA is less than about 6 pg/mg. In some embodiments, the residual plasmid DNA in the purified mRNA is less than about 7 pg/mg. In some embodiments, the residual plasmid DNA in the purified mRNA is less than about 8 pg/mg. In some embodiments, the residual plasmid DNA in the purified mRNA is less than about 9 pg/mg. In some embodiments, the residual plasmid DNA in the purified mRNA is less than about 10 pg/mg. In some embodiments, the residual plasmid DNA in the purified mRNA is less than about 11 pg/mg. In some embodiments, the residual plasmid DNA in the purified mRNA is less than about 12 pg/mg.

The mRNA can be complexed to the pH sensitive protonatable or ionizable lipids described herein by admixing the mRNA and the pH sensitive protonatable or ionizable lipids or corresponding disulfide oligomer or polymer. The pH of the reaction can be modified to convert the amino groups present on the pH sensitive protonatable or ionizable lipids described herein to cationic groups. For example, the pH can be adjusted to protonate the amino group. With the presence of cationic groups on the compound, the nucleic acid can electrostatically bond (i.e., complex) to the compound. In one aspect, the pH is from 1 to 7.4.

In another aspect, the N/P ratio of the pH sensitive protonatable or ionizable lipids complexed with nucleic acids can be from 0.5 to 100, where N is the number of nitrogen atoms (e.g., amines) present on the compounds that can form a positive charge and P is the number of phosphate groups present on the nucleic acids. Thus, by modifying the pH sensitive protonatable or ionizable lipids with the appropriate number of amino groups in the head group, it is possible to tailor the bonding (e.g., type and strength of bond) between the nucleic acid and the pH sensitive protonatable or ionizable lipids. The N/P ratio can be adjusted depending on the cell type to which the nucleic acid is to be delivered. In some embodiments, the N/P ratio can be at least about 6, at least about 10, or at least about 15. In other embodiments, the N/P ratio can be from about 6 to about 20, about 10 to about 20, about 12 to about 20, or about 6 about 14. In still other embodiments, the N/P ratio can be about 2 to about 20, about 2 to about 18, about 2 to about 16, about 2 to about 14, about 2 to about 12, or about 4 to about 12.

In one aspect, the stabilized lipid nanoparticle can have an average particle diameter of about 100 nm to less than about 500 nm, about 100 nm to about 400 nm, about 100 nm to about 300 nm, or about 100 nm to about 200 nm. In another aspect, the stabilized lipid nanoparticle can have a diameter of about 1000 nanometers or less, for example, about 50 nm to about 200 nm, about 60 nm to about 180 nm, about 70 nm to about 160 nm, about 80 nm to about 140 nm, or about 90 nm to about 120 nm.

In other aspects, the lipid nanoparticles described herein can be designed to escape endosomal and/or lysosomal compartments at the endosomal-lysosomal pH. For example, the pH sensitive protonatable or ionizable lipids forming nanoparticles with nucleic acids can be designed such that its structure and amphiphilicity changes at endosomal-lysosomal pH (5.0-6.0) and disrupts endosomal-lysosomal membranes, which allows entry of the nanoparticle into the cytoplasm. In one aspect, the ability of specific endosomal-lysosomal membrane disruption of the pH sensitive protonatable or ionizable lipids described herein can be tuned by modifying their pH sensitive amphiphilicity by altering the number and structure of protonatable amines and lipophilic groups. For example, decreasing the number of protonatable amino groups can reduce the amphiphilicity of a nanoparticle produced by the pH sensitive protonatable or ionizable lipids at neutral pH. In one aspect, the pH sensitive protonatable or ionizable lipids described herein have 1 to 50, 1 to 40, 1 to 30, 1 to 20, 1 to 10, 1 to 8, 1 to 6, 1 to 4, or 2 protonatable amino or substituted amino groups. The pH-sensitive amphiphilicity of the pH sensitive protonatable or ionizable lipids and nanoparticles produced by the pH sensitive protonatable or ionizable lipids can be used to fine-tune the overall pKa of the nanoparticle. Low amphiphilicity of the nanoparticles at physiological pH can minimize non-specific cell membrane disruption and nonspecific tissue uptake of the nucleic acid. In certain aspects, it is desirable that the lipid nanoparticles have low amphiphilicity at the physiological pH and high amphiphilicity at the endosomal-lysosomal pH, which will only cause selective endosomal-lysosomal membrane disruption with the nanoparticles.

The amount of a therapeutic and/or prophylactic in a nanoparticle composition comprising a plurality of stabilized lipid nanoparticles described herein may depend on the size, composition, desired target and/or application, or other properties of the stabilized lipid nanoparticles as well as on the properties of the therapeutic and/or prophylactic. For example, the amount of an mRNA useful in a nanoparticle composition may depend on the size, sequence, and other characteristics of the mRNA. The relative amounts of a therapeutic and/or prophylactic and other elements (e.g., pH sensitive protonatable or ionizable lipids) in a nanoparticle composition may also vary. In some embodiments, the wt/wt ratio of the pH sensitive protonatable or ionizable lipids to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the pH sensitive protonatable or ionizable lipids to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

Nanoparticle compositions may be formulated in whole or in part as pharmaceutical compositions. Pharmaceutical compositions may include one or more nanoparticle compositions. For example, a pharmaceutical composition may include one or more nanoparticle compositions including one or more different therapeutic and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21.sup.st Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more nanoparticle compositions.

In certain embodiments, the nanoparticle composition includes cryoprotectant, such as sucrose, that is provided in the pharmaceutical composition at an amount effective to enhance the stability of the composition when cryopreserved. However, any cryoprotectant will suffice (e.g., trehalose). The concentration of the cryo-protectant can be from 4%-32% v/v.

In certain embodiments, the nanoparticle compositions and/or pharmaceutical compositions can be refrigerated or frozen for storage and/or shipment, e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.).

In certain embodiments, a method of increasing stability of the nanoparticle compositions and/or pharmaceutical compositions can include storing the nanoparticle compositions and/or pharmaceutical compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C. For example, the lipid nanoparticle composition described herein and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.).

In certain embodiments, the pharmaceutical composition can include a nanoparticle composition disclosed herein and a pharmaceutically acceptable carrier selected from one or more of Tris, an acetate (e.g., sodium acetate), a citrate (e.g., sodium citrate), saline, PBS, and sucrose. In certain embodiments, the carrier may be at a concentration of 1-100 mM (e.g., including but not limited to any numerical value or range within the range of 1-100 mM such as 1, 2, 3, 4, . . . 97, 98, 99, 100, 10-90 mM, 20-80 mM, 30-70 mM and so on).

Lipid nanoparticle compositions and/or pharmaceutical compositions including one or more nanoparticle compositions may be administered to any patient or subject, including those patients or subjects that may benefit from a therapeutic effect provided by the delivery of a therapeutic and/or prophylactic to one or more particular cells, tissues, organs, or systems or groups thereof. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition described herein may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

The lipid nanoparticles described herein can be administered to a subject using techniques known in the art. For example, pharmaceutical compositions can be prepared with the complexes. It will be appreciated that the actual preferred amounts of the complex in a specified case will vary according to the specific compound being utilized, the particular compositions formulated, the mode of application, and the particular sites and subject being treated. Dosages for a given host can be determined using conventional considerations, e.g., by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians' Desk Reference, Barnhart Publishing (1999).

The mRNA/lipid nanoparticles described herein can be used for treating patients. The mRNA encapsulated in lipid nanoparticles can be delivered to cells or subjects in vivo and encode a protein or polypeptide. Accordingly, in certain embodiments, mRNA/lipid nanoparticles can used in a therapeutic composition comprising mRNA that encode a peptide or polypeptide for use in the delivery to or treatment of, for example, the lung of a subject or a lung cell, liver of a subject or a liver cell, cardiovasculature of a subject or a cardiovascular cell, muscle of a subject or a muscle cell, nervous system of a subject or a nervous system cell, blood or bone marrow of a subject or a blood or bone marrow cell, kidney of a subject or a kidney cell, or eye of a subject or an eye cell.

For example, the mRNA/lipid nanoparticles can include mRNA that encode for ATP-binding cassette sub-family A member 4 (ABCA4), retinoschisin protein, retinal pigment epithelium-specific 65 kDa (RPE65) protein, or centrosomal protein of 290 kDa (CEP290).

In other embodiments, the mRNA/lipid nanoparticles described herein can include mRNA that encodes a peptide or polypeptide for use in the delivery of or treatment with a vaccine for a subject or a cell of a subject. For example, in some embodiments the mRNA/lipid nanoparticles described herein provides a method for producing a therapeutic composition having mRNA that encode for an antigen from an infectious agent, such as a virus, coronavirus, COVID virus, influenza virus, respiratory syncytial virus, rabies virus, cytomegalovirus, rotavirus, hepatitis A virus, hepatitis B virus, or hepatitis C virus, human papillomavirus herpes simplex virus, such as herpes simplex virus 1 or herpes simplex virus 2. In certain embodiments the present invention provides a method for producing a therapeutic composition having mRNA that encode for an antigen from a human immunodeficiency virus, such as human immunodeficiency virus type 1 or human immunodeficiency virus type 2, human metapneumovirus, human parainfluenza virus, human parainfluenza virus type 1, human parainfluenza virus type 2, human parainfluenza virus type 3, malaria virus, zika virus, or chikungunya virus.

To facilitate expression of mRNA in vivo, compositions encapsulating mRNA can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

The mRNA/lipid nanoparticles described herein, and compositions containing the same, may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical, and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

The mRNA/lipid nanoparticles described herein can be administered via intravenous delivery, subcutaneous delivery, oral delivery, subdermal delivery, ocular delivery, intratracheal injection pulmonary delivery (e.g., nebulization), intramuscular delivery, intrathecal delivery, or intraarticular delivery.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal.

In some embodiments, a composition comprising the mRNA/lipid nanoparticles is administered by intravenous delivery. In some embodiments, a composition comprising the mRNA/lipid nanoparticles is administered by subcutaneous delivery. In some embodiments, a composition comprising the mRNA/lipid nanoparticles is administered by oral delivery. In some embodiments, a composition comprising the mRNA/lipid nanoparticles is administered by subdermal delivery. In some embodiments, a composition comprising the mRNA/lipid nanoparticles is administered by ocular delivery. In some embodiments, a composition comprising the mRNA/lipid nanoparticles is administered by intratracheal delivery. In some embodiments, a composition comprising the mRNA/lipid nanoparticles is administered by pulmonary delivery. In some embodiments, a composition comprising the mRNA/lipid nanoparticles is administered by nebulization delivery. In some embodiments, a composition comprising the mRNA/lipid nanoparticles is administered by intramuscular delivery. In some embodiments, a composition comprising the mRNA/lipid nanoparticles is administered by intrathecal delivery. In some embodiments, a composition comprising the mRNA/lipid nanoparticles is administered by intraarticular delivery. In some embodiments, a composition comprising the mRNA/lipid nanoparticles is administered by rectal delivery. In some embodiments, a composition comprising the mRNA/lipid nanoparticles is administered by vaginal delivery. In some embodiments, a composition comprising the mRNA/lipid nanoparticles is administered by transmucosal delivery. In some embodiments, a composition comprising the mRNA/lipid nanoparticles is administered by intestinal delivery. In some embodiments, a composition comprising the mRNA/lipid nanoparticles is administered by parental delivery. In some embodiments, a composition comprising the mRNA/lipid nanoparticles is administered by intradermal delivery. In some embodiments, a composition comprising the mRNA/lipid nanoparticles is administered by subcutaneous delivery. In some embodiments, a composition comprising the mRNA/lipid nanoparticles is administered by intraventricular delivery. In some embodiments, a composition comprising the mRNA/lipid nanoparticles is administered by intraperitoneal delivery. In some embodiments, a composition comprising the mRNA/lipid nanoparticles is administered by intranasal delivery.

In some embodiments, the intramuscular administration is to a muscle selected from the group consisting of skeletal muscle, smooth muscle and cardiac muscle. In some embodiments, the administration results in delivery of the mRNA to a muscle cell. In some embodiments, the administration results in delivery of the mRNA to a hepatocyte (i.e., liver cell). In a particular embodiment, the intramuscular administration results in delivery of the mRNA to a muscle cell.

Additional teaching of pulmonary delivery and nebulization are described in published U.S. Application No. US 2018/0125989 and published U.S. Application No. US 2018/0333457, each of which is incorporated by reference in its entirety.

Alternatively or additionally, the mRNA/lipid nanoparticles and compositions described herein may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

Provided methods contemplate single as well as multiple administrations of a therapeutically effective amount of the therapeutic agents (e.g., mRNA) described herein. Therapeutic agents can be administered at regular intervals, depending on the nature, severity and extent of the subject's condition. In some embodiments, a therapeutically effective amount of the therapeutic agents (e.g., mRNA) of the present invention may be administered intrathecally periodically at regular intervals (e.g., once every year, once every six-months, once every five-months, once every three-months, bimonthly (once every two-months), monthly (once every month), biweekly (once every two-weeks), twice a month, once every 30-days, once every 28-days, once every 14-days, once every 10-days, once every 7-days, weekly, twice a week, daily, or continuously).

In some embodiments, provided mRNA/lipid nanoparticles and/or compositions are formulated such that they are suitable for extended-release of the mRNA contained therein. Such extended-release compositions may be conveniently administered to a subject at extended dosing intervals. For example, in one embodiment, the compositions of the present invention are administered to a subject twice a day, daily, or every other day. In a preferred embodiment, the compositions of the present invention are administered to a subject twice a week, once a week, once every 7-days, once every 10-days, once every 14-days, once every 28-days, once every 30-days, once every two-weeks, once every three-weeks, or more-preferably once every four-weeks, once-a-month, twice-a-month, once every six-weeks, once every eight-weeks, once every other month, once every three-months, once every four-months, once every six-months, once every eight-months, once every nine-months, or annually. Also contemplated are compositions and liposomes that are formulated for depot administration (e.g., intramuscularly, subcutaneously, intravitreally) to either deliver or release therapeutic agent (e.g., mRNA) over extended periods of time. Preferably, the extended-release means employed are combined with modifications made to the mRNA to enhance stability.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating a disease or disorder). For example, a therapeutically effective amount may be an amount sufficient to achieve a desired therapeutic and/or prophylactic effect. Generally, the amount of a therapeutic agent (e.g., mRNA) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific protein employed; the duration of the treatment; and like factors as is well known in the medical arts.

In some embodiments, the therapeutically effective dose ranges from about 0.005 mg/kg body weight to 500 mg/kg body weight, e.g., from about 0.005 mg/kg body weight to 400 mg/kg body weight, from about 0.005 mg/kg body weight to 300 mg/kg body weight, from about 0.005 mg/kg body weight to 200 mg/kg body weight, from about 0.005 mg/kg body weight to 100 mg/kg body weight, from about 0.005 mg/kg body weight to 90 mg/kg body weight, from about 0.005 mg/kg body weight to 80 mg/kg body weight, from about 0.005 mg/kg body weight to 70 mg/kg body weight, from about 0.005 mg/kg body weight to 60 mg/kg body weight, from about 0.005 mg/kg body weight to 50 mg/kg body weight, from about 0.005 mg/kg body weight to 40 mg/kg body weight, from about 0.005 mg/kg body weight to 30 mg/kg body weight, from about 0.005 mg/kg body weight to 25 mg/kg body weight, from about 0.005 mg/kg body weight to 20 mg/kg body weight, from about 0.005 mg/kg body weight to 15 mg/kg body weight, from about 0.005 mg/kg body weight to 10 mg/kg body weight.

In some embodiments, the therapeutically effective dose is greater than about 0.1 mg/kg body weight, greater than about 0.5 mg/kg body weight, greater than about 1.0 mg/kg body weight, greater than about 3 mg/kg body weight, greater than about 5 mg/kg body weight, greater than about 10 mg/kg body weight, greater than about 15 mg/kg body weight, greater than about 20 mg/kg body weight, greater than about 30 mg/kg body weight, greater than about 40 mg/kg body weight, greater than about 50 mg/kg body weight, greater than about 60 mg/kg body weight, greater than about 70 mg/kg body weight, greater than about 80 mg/kg body weight, greater than about 90 mg/kg body weight, greater than about 100 mg/kg body weight, greater than about 150 mg/kg body weight, greater than about 200 mg/kg body weight, greater than about 250 mg/kg body weight, greater than about 300 mg/kg body weight, greater than about 350 mg/kg body weight, greater than about 400 mg/kg body weight, greater than about 450 mg/kg body weight, greater than about 500 mg/kg body weight. In a particular embodiment, the therapeutically effective dose is 1.0 mg/kg. In some embodiments, the therapeutically effective dose of 1.0 mg/kg is administered intramuscularly or intravenously.

Provided stabilized lipid nanoparticles and compositions may be administered to any desired tissue. In some embodiments, the mRNA delivered by provided lipid nanoparticles or compositions is expressed in the tissue in which the lipid nanoparticles and/or compositions were administered. In some embodiments, the mRNA delivered is expressed in a tissue different from the tissue in which the lipid nanoparticles and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

In some embodiments, administering the provided composition results in an increased mRNA expression level in a biological sample from a subject as compared to a baseline expression level before treatment. Typically, the baseline level is measured immediately before treatment. Biological samples include, for example, whole blood, serum, plasma, urine and tissue samples (e.g., muscle, liver, skin fibroblasts). In some embodiments, administering the provided composition results in an increased mRNA expression level by at least about 10%, 20%, 30%, 40%, 50%, 60%, 70%, 80%, 90%, or 95% as compared to the baseline level immediately before treatment. In some embodiments, administering the provided composition results in an increased mRNA expression level as compared to an mRNA expression level in subjects who are not treated.

According to various embodiments, the timing of expression of delivered mRNA can be tuned to suit a particular medical need. In some embodiments, the expression of the protein encoded by delivered mRNA is detectable 1, 2, 3, 6, 12, 24, 48, 72, and/or 96 hours after administration of provided stabilized lipid nanoparticles and/or compositions. In some embodiments, the expression of the protein encoded by delivered mRNA is detectable one-week, two-weeks, and/or one-month after administration.

The following example is for the purpose of illustration only and is not intended to limit the scope of the claims, which are appended hereto.

### Example 1

### Preparation of ECO/mRNA Nanoparticles

ECO/mRNA nanoparticles were prepared through self-assembly of ECO and mRNA in aqueous solution. Specifically, ECO stock solution (50 mM in ethanol) and mRNA stock solution (1 μg/μL) were mixed and shaken in an aqueous solution at a predetermined concentration of 100 ng/μL, and N/P ratios (4, 6, 8, 10 and 12) for 30 min at room temperature to give ECO/mRNA nanoparticles.

### Characterization of ECO/mRNA Nanoparticles

Condensation of mRNA with ECO into nanoparticles was verified by agarose gel electrophoresis prepared in a 0.7% agarose gel run at 120 V for 25 min. Size and zeta potential of the nanoparticles were determined using dynamic light scattering (DLS) with an Anton Paar Litesizer 500 (Anton Paar USA, Ashland, VA).

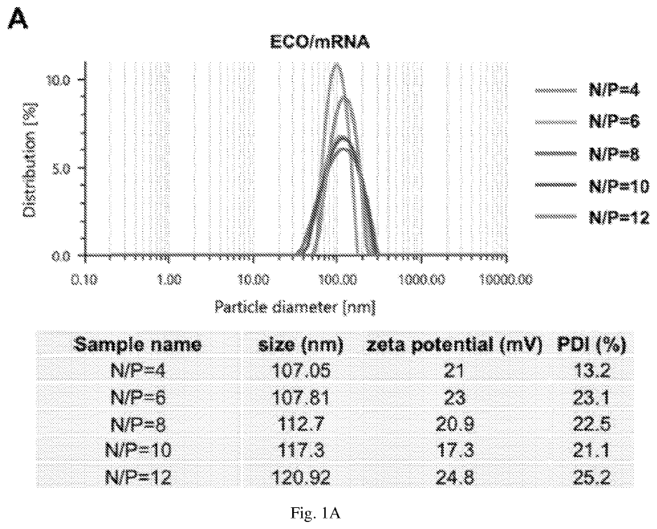

The Characterization of ECO/mRNA nanoparticles formulated at different N/P ratios were shown in FIG. 1. ECO and mRNA could form stable nanoparticles with sizes around 120 nm, positive zeta potentials of around 20 mV, and PDIs around 25%, FIG. 1A. mRNA encapsulation was also confirmed by agarose gel electrophoresis FIG. 1B. ECO could efficiently encapsulate mRNA for all the N/P ratios tested, indicating stable nanoparticles were formed.

### ECO/mRNA can Transfect Cells and Result in High Gene Expression

ECO/mRNA nanoparticles were tested in vitro transfection efficiency in ARPE-19 cells. ECO/mRNA nanoparticles were formulated at N/P ratios of 4,6,8,10,12 with mRNA that encodes EGFP. The final concentration of mRNA in the ECO/mRNA nanoparticle solution was 100 ng/μL. ARPE-19 cells were seeded at 40,000 cells/well concentration on a 12-well plate. After the cells reached about 40-50% confluency, the nanoparticles were added to each well. GFP expression was evaluated using confocal microscopy 24 h and 48 h. The GFP fluorescent intensity was evaluated using a spectrophotometer and the cell viability was evaluated using CCK-8 assay at 48 h.

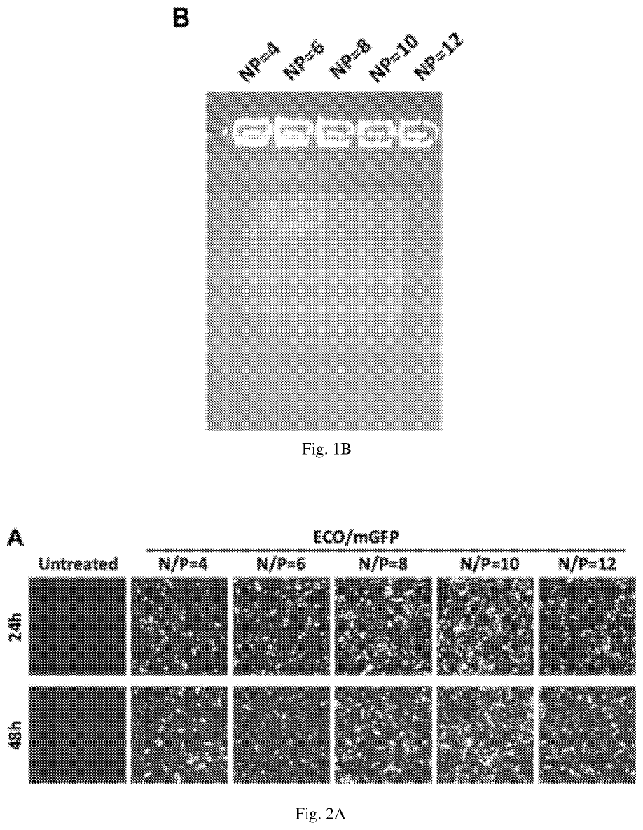

As demonstrated in FIG. 2, stronger GFP expressions were observed across all the time points from ECO/mRNA nanoparticles of all N/P ratios (FIG. 2A). There was an increasing trend of GFP expressions with the N/P ratios (FIG. 2A, B). Transfection did induce lower cell viability, which could be resolved through PEGylation (FIG. 1C). Therefore, ECO/mRNA nanoparticles can transfect ARPE-19 cells and result in high gene expression with limited cell viability which could be resolved through PEGylation of the nanoparticles.

ECO/mRNA nanoparticles were also tested in vitro transfection efficiency in ARPE-19 cells for different mRNA doses. ECO/mRNA nanoparticles were formulated at N/P ratios of 8 with mRNA that encodes EGFP at different doses of 10, 25, 50, 100, and 200 ng. ARPE-19 cells were seeded at 40,000 cells/well concentration on a 12-well plate. After the cells reached about 40-50% confluency, the nanoparticles were added to each well. GFP expression was evaluated using confocal microscopy 24 h and 48 h. The GFP fluorescent intensity was evaluated using a spectrophotometer.

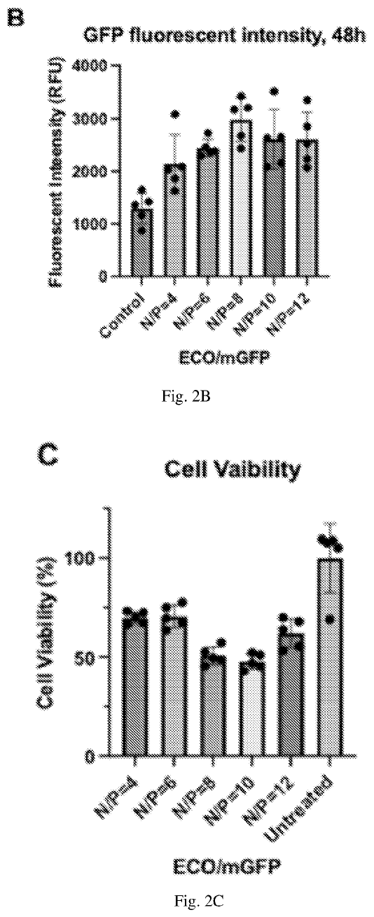

As demonstrated in FIG. 3, GFP expressions were observed across all the time points for all the mRNA doses (FIG. 3A). There was an increasing trend of GFP expressions with the doses (FIGS. 3A, B).

ECO/mRNA nanoparticles were also tested in vitro transfection efficiency in another cell line (HEK293T cells). ECO/mRNA nanoparticles were formulated at N/P ratio of 8 with mRNA that encodes EGFP. The final concentration of mRNA in the ECO/mRNA nanoparticle solution was 100 ng/μL. HEK293T cells were seeded at 40,000 cells/well concentration on a 12-well plate. After the cells reached about 40-50% confluency, the nanoparticles were added to each well. GFP expression was evaluated using confocal microscopy 24, 48, 72 h.

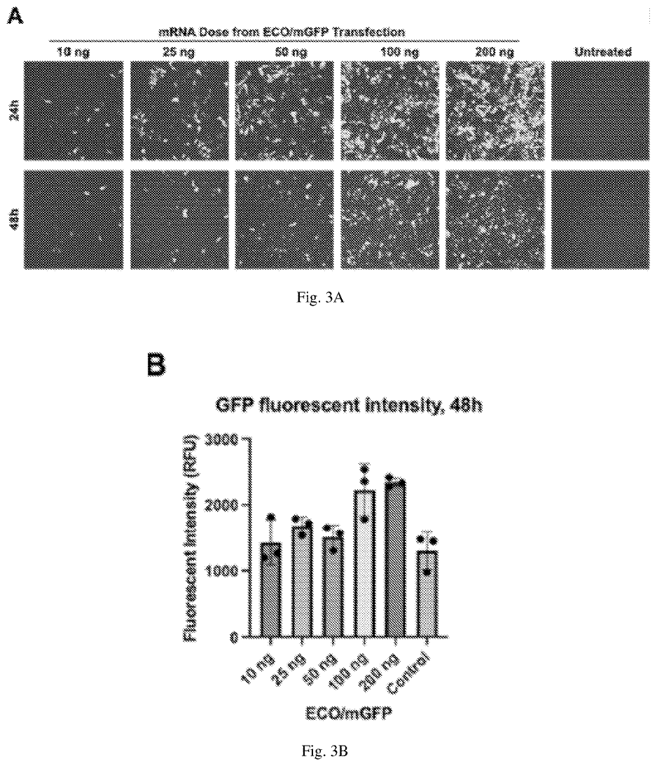

As demonstrated in FIG. 4, stronger GFP expressions were observed across all the time points from ECO/mRNA nanoparticles than Lipofectamine.

ECO can Form Stable Nanoparticle Directly with mRNA and PEGylation of the ECO/mRNA Nanoparticle can Enhance its Stability and Viability

ECO/mRNA nanoparticles were formulated at different N/P ratios of 4,6,8,10 and 12 with mRNA. The final concentration of mRNA in the ECO/mRNA nanoparticle solution was 100 ng/μL. PEG-MAL (0.625 mM) was added to the ECO/mRNA nanoparticle complex solution at 2.5 mol % for PEGylation. The mixture was vortexed for at least 15~30 min allowing complete PEGylation. Sucrose (5%) was added as an excipient for storage. Condensation of the mRNA with ECO into nanoparticles was verified by agarose gel electrophoresis prepared in a 0.7% agarose gel run at 100 V for 25 min. Size and zeta potential of the nanoparticles were determined using dynamic light scattering (DLS) with an Anton Paar Litesizer 500 (Anton Paar USA, Ashland, VA).

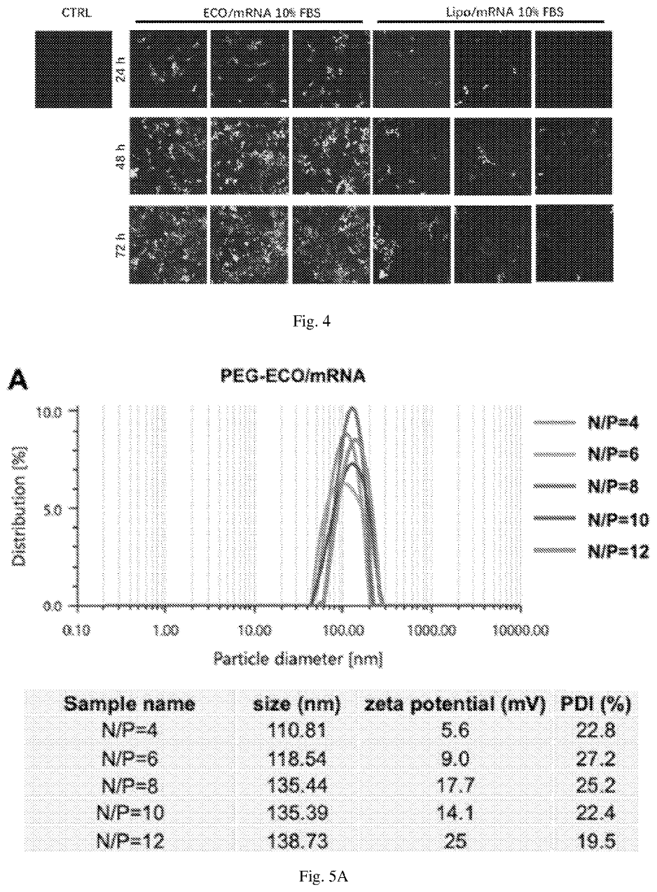

The Characterization of PEGylated ECO/mRNA nanoparticles formulated at different N/P ratios were shown in FIG. 5. PEGylated ECO and mRNA could form stable nanoparticles with sizes around 130 nm, increased positive zeta potentials with the N/P ratios of from 5.6 to 25 mV, and PDIs around 25%, FIG. 5A. mRNA encapsulation was also confirmed by agarose gel electrophoresis FIG. 5B. PEGylated ECO/mRNA nanoparticle formulations could efficiently encapsulate mRNA for all the N/P ratios tested, indicating stable nanoparticles were formed.

PEGylated ECO/mRNA nanoparticles were tested in vitro transfection efficiency in ARPE-19 cells. PEGylated ECO/mRNA nanoparticles were formulated at N/P ratios of 4,6,8,10,12 with mRNA that encodes EGFP. The final concentration of mRNA in the PEGylated ECO/mRNA nanoparticle solution was 100 ng/μL. ARPE-19 cells were seeded at 40,000 cells/well concentration on a 12-well plate. After the cells reached about 40-50% confluency, the nanoparticles were added to each well. GFP expression was evaluated using confocal microscopy 24 h and 48 h. The GFP fluorescent intensity was evaluated using a spectrophotometer and the cell viability was evaluated using CCK-8 assay at 48 h.

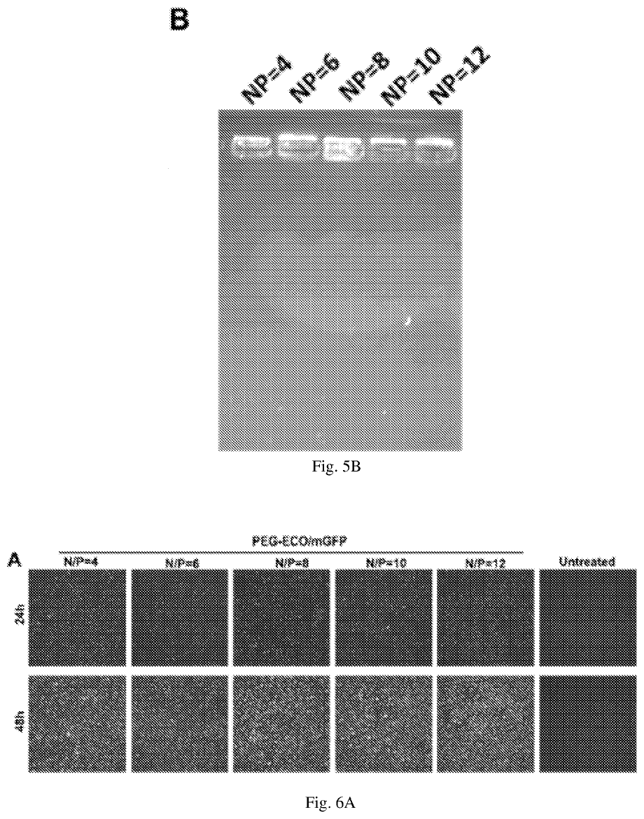

As demonstrated in FIG. 6, obvious GFP expressions were observed across all the time points from PEGylated ECO/mRNA nanoparticles of all N/P ratios (FIG. 6A). There was a slight increasing trend of GFP expressions with the N/P ratios (FIG. 6A, B). The GFP expression is higher at 48 h than 24 h. Transfection did not induce any cell toxicity, (FIG. 6C).

PEGylated ECO/mRNA nanoparticles were also tested in vitro transfection efficiency in ARPE-19 cells for different mRNA doses. PEGylated ECO/mRNA nanoparticles were formulated at N/P ratios of 8 with mRNA that encodes EGFP at different doses of 10, 25, 50, 100, and 200 ng. ARPE-19 cells were seeded at 40,000 cells/well concentration on a 12-well plate. After the cells reached about 40-50% confluency, the nanoparticles were added to each well. GFP expression was evaluated using confocal microscopy 24 h and 48 h. The GFP fluorescent intensity was evaluated using a spectrophotometer.

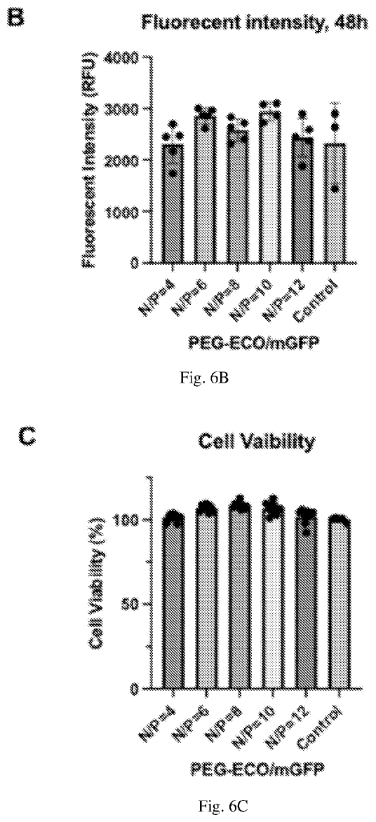

As demonstrated in FIG. 7, GFP expressions were observed across all the time points for all the mRNA doses (FIG. 7A). There was an increasing trend of GFP expressions with the doses (FIG. 7A, B). GFP expression was higher for 48 h than 24 h (FIG. 7A).

PEGylated ECO/mRNA nanoparticles were tested stability in PBS incubation and after 2 freeze-thaw cycles (storage under −80° C.). PEGylated ECO/mRNA nanoparticles were formulated at N/P ratio of 8 with mRNA that. The final concentration of mRNA in the PEGylated ECO/mRNA nanoparticle solution was 100 ng/μL. PEGylated ECO/mRNA nanoparticles were incubated in PBS for 24 h and 2 weeks. The stability of the PEGylated ECO/mRNA nanoparticles were also tested after 2 freeze-thaw cycles (−80° C. storage). DLS was used to evaluate the size and distributions of the nanoparticle.

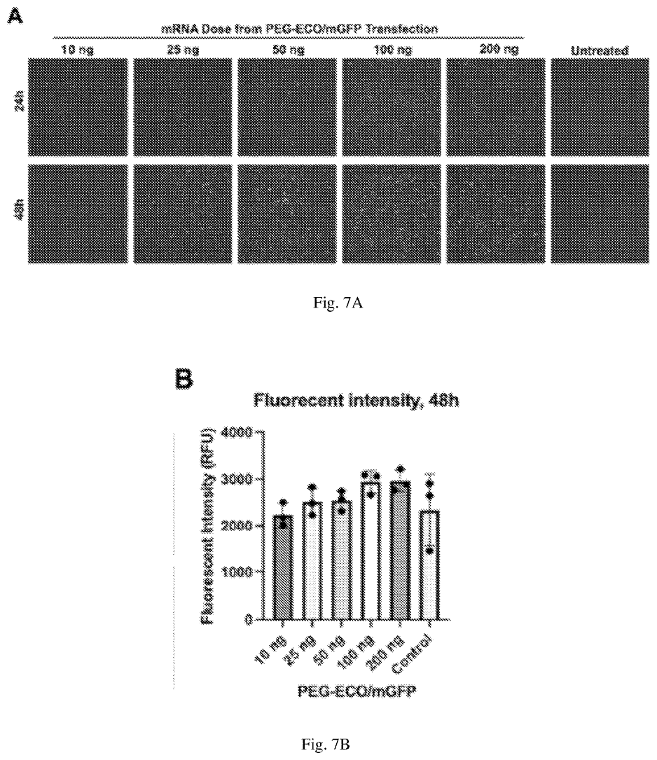

Results demonstrated in FIG. 8 has shown that PEGylated ECO/mRNA nanoparticles can from stable nanoparticle formulations. The nanoparticle could maintain its size distribution, size and PDI after both PBS incubation (FIG. 8A) and freeze-thaw cycles (FIG. 8B). Therefore, PEGylated ECO/mRNA demonstrated excellent storage ability and ready to be used with PBS dilution.

### PEGylated ECO/mRNA Nanoparticles can Induce High Expression in Mice

PEGylated ECO/mRNA nanoparticles were also tested in vivo transfection efficiency in nude mice. PEGylated (PEG-RGD) ECO/mRNA nanoparticles were formulated at N/P ratios of 8 with mRNA that encodes EGFP and luciferase at a concentration of 100 ng/ng/μL. Nude mice were injected in the leg muscle with 5 g of mRNA (mEGFP or mLuc). GFP and luciferase expression were imaged 6 h after injections.

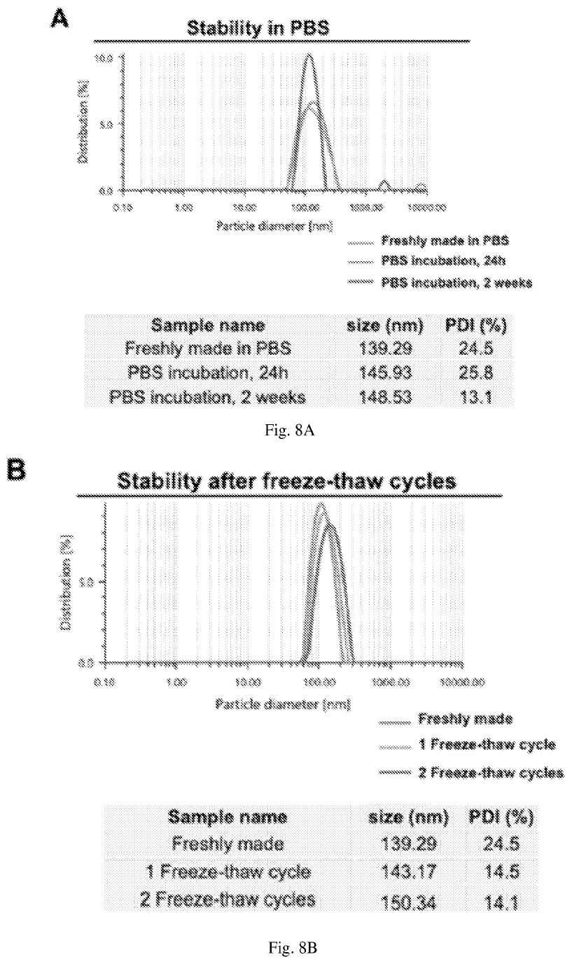

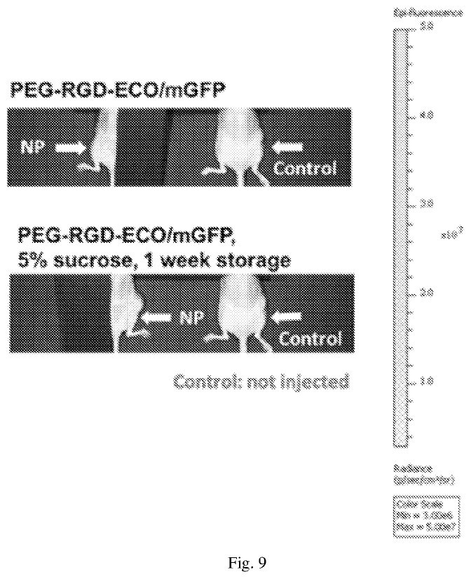

Results demonstrated significant GFP expression 6 h after muscle injection of PEGylated ECO/mGFP nanoparticles (FIGS. 9 and 10). No significant difference has been observed between freshly made PEGylated ECO/mGFP nanoparticles and nanoparticles after 1 week storage under −80° C., indicating excellent stability of PEGylated ECO/mRNA nanoparticles with no sacrifice of efficiency after storage and freeze-thaw cycles.

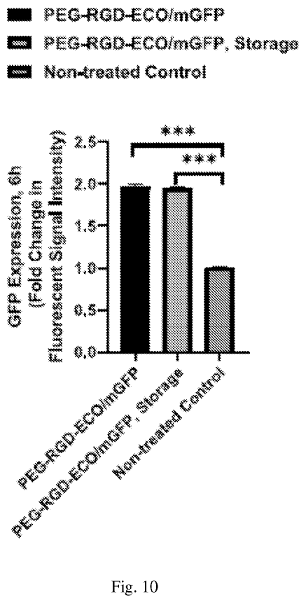

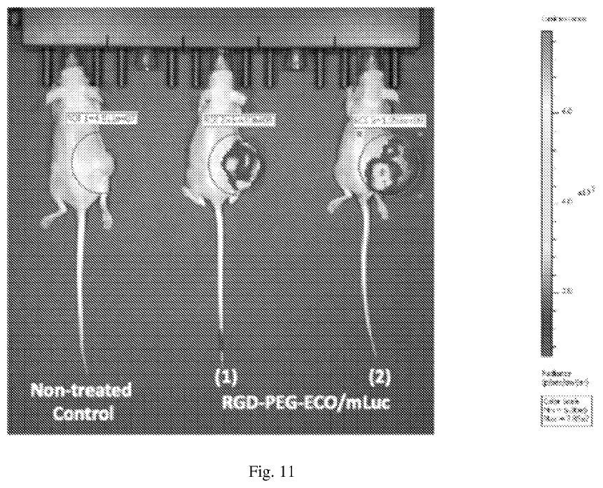

Similar results demonstrated significant luciferase expression 6 h after muscle injection of PEGylated ECO/mLuc nanoparticles (FIGS. 11 and 12). Therefore, PEGylated ECO/mRNA nanoparticles demonstrated excellent in vivo transfection efficiency.

ECO/mRNA Nanoparticle can be Formulated with Hyaluronic Acid (HA) as Surface Modification

ECO/mRNA nanoparticles can be further formulated with hyaluronic acid (HA) as surface modification. As previously described, ECO/mRNA nanoparticles were formulated at N/P ratio of 8 with mRNA that encodes EGFP. The final concentration of mRNA in the ECO/mRNA nanoparticle solution was 100 ng/μL. The ECO stock solution (50 mM in ethanol) and mRNA stock solution (0.5 mg/mL) at predetermined amounts based on the N/P ratio were mixed and vortexed for 15 min at room temperature. After the ECO/mRNA nanoparticle was formed. Hyaluronic acid (Mw=8,000~15,000 Da) was added to the ECO/mRNA nanoparticle solution at 2.5 mol % and 5 mol % regarding the amount of ECO. Then the solution was shaken for another 15 minutes. Condensation of the mRNA with ECO and HA into nanoparticles was verified by agarose gel electrophoresis prepared in a 0.7% agarose gel run at 100 V for 25 min. Size and zeta potential of the nanoparticles were determined using dynamic light scattering (DLS) with an Anton Paar Litesizer 500 (Anton Paar USA, Ashland, VA).

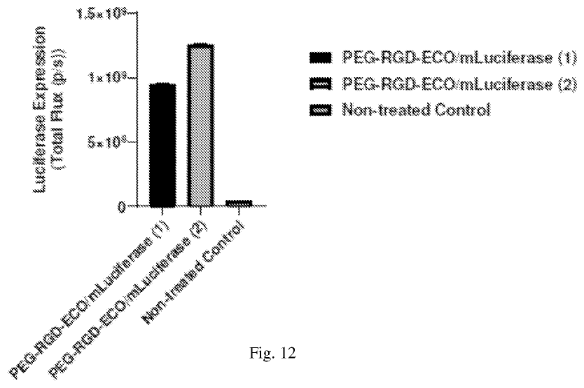

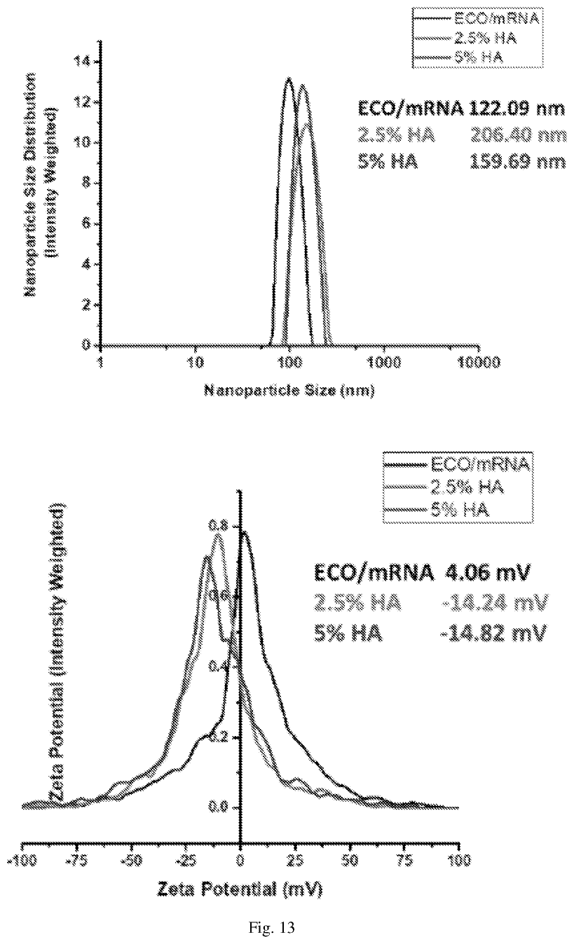

The results of ECO/mRNA nanoparticle formulations with hyaluronic acid were shown in FIGS. 13 and 14. ECO and mRNA could form stable nanoparticle with HA at both 2.5 mol % and 5 mol % with a size around 200 nm (FIG. 13). Zeta potential after adding HA to the ECO/mRNA nanoparticles became negative, due to the negative surface charge of HA (FIG. 14). The mRNA encapsulation was also confirmed by agarose gel electrophoresis FIG. 14. ECO could efficiently encapsulate mRNA both before and after HA modification at 2.5 mol % and 5 mol %.

### Hyaluronic Acid (HA) Modified ECO/mRNA Nanoparticles can Transfect Cells and Result in High Gene Expression

HA modified ECO/mRNA nanoparticles were tested in vitro transfection efficiency in ARPE-19 cells. ECO/mRNA nanoparticles were formulated at N/P ratio of 8 with mRNA that encodes EGFP. The final concentration of mRNA in the ECO/mRNA nanoparticle solution was 100 ng/μL. HA was used for surface modification in the formulation at both 2.5 mol % and 5 mol % regarding the amount of ECO. ARPE-19 cells were seeded at 40,000 cells/well concentration on a 12-well plate. After the cells reached about 40-50% confluency, the nanoparticles were added to each well. GFP expression was evaluated using confocal microscopy at 48 h.

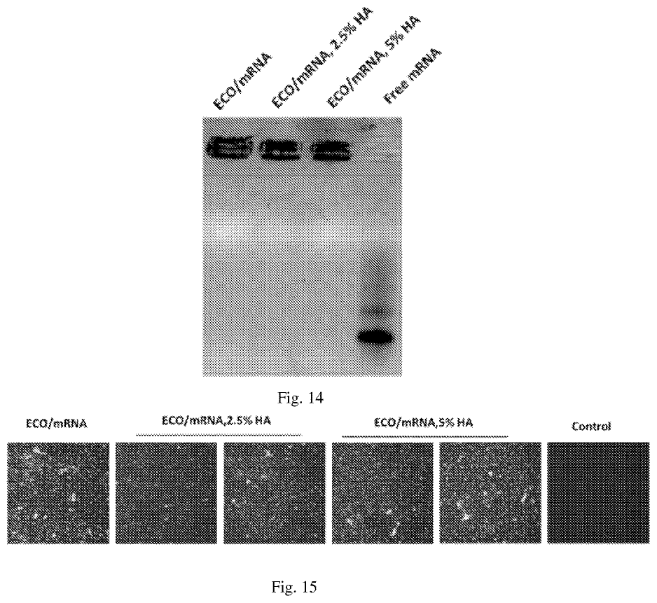

As demonstrated in FIG. 15, strong GFP expressions were observed in ARPE-19 cells 48 h after transfections using ECO/mRNA nanoparticles and HA modified ECO/mRNA nanoparticles. Therefore, modification of ECO/mRNA nanoparticles with HA can result in stable nanoparticle formulations and excellent gene expressions.

ECO/mRNA Nanoparticle can be Formulated with Functionalized Dextran (Dextran-Mal) as Surface Modification

ECO/mRNA nanoparticles can also be formulated with functionalized dextran (dextran-mal, 5k Da) as surface modification. As previously described, ECO/mRNA nanoparticles were formulated at N/P ratio of 8 with mRNA. The final concentration of mRNA in the ECO/mRNA nanoparticle solution was 100 ng/μL. The ECO stock solution (50 mM in ethanol) and mRNA stock solution (0.5 mg/mL) at predetermined amounts based on the N/P ratio were mixed and vortexed for 15 min at room temperature. After the ECO/mRNA nanoparticle was formed. Dextran-mal (Mw=5k Da) was added to the ECO/mRNA nanoparticle solution at 2.5 mol %, 5 mol %, 10 mol %, and 15 mol % regarding the amount of ECO. Then the solution was shaken for another 15 minutes. Size and zeta potential of the nanoparticles were determined using dynamic light scattering (DLS) with an Anton Paar Litesizer 500 (Anton Paar USA, Ashland, VA).

TABLE 1
Functionalized Dextran-mal modified ECO/mRNA
nanoparticle formulations. DLS measurements
of size, zeta potential and PDI.

| | Dextran & | Size | Zeta | PDI |
|---|---|---|---|---|
| Sample name | weight | (nm) | potential (mV) | (%) |
| ECO/mRNA, 2.5% Dex | 2.50% | 96.91 | 5.6 | 24.1 |
| ECO/mRNA, 5% Dex | 5% | 127.29 | 8.6 | 26.8 |
| ECO/mRNA, 10% Dex | 10% | 143.52 | 22.5 | 23.5 |
| ECO/mRNA, 15% Dex | 15% | 127.39 | 30.3 | 15.9 |

ECO and mRNA could form stable nanoparticle with functionalized dextran-mal at 2.5, 5, 10 and 15 mol % with sizes less than 150 nm, zeta potentials of 5.6 mV-30.3 mV and PDIs around 25% (Table 1).

### Functionalized Dextran (Dextran-Mal) Modified ECO/mRNA Nanoparticles can Transfect Cells And Result in High Gene Expression

Dextran-mal modified ECO/mRNA nanoparticles were tested in vitro transfection efficiency in ARPE-19 cells. ECO/mRNA nanoparticles were formulated at N/P ratio of 8 with mRNA that encodes EGFP. The final concentration of mRNA in the ECO/mRNA nanoparticle solution was 100 ng/μL. Dextran-mal was used for surface modification in the formulation at 10 mol % regarding the amount of ECO. ARPE-19 cells were seeded at 40,000 cells/well concentration on a 12-well plate. After the cells reached about 40-50% confluency, the nanoparticles were added to each well. GFP expression was evaluated using confocal microscopy at 24 and 48 h.

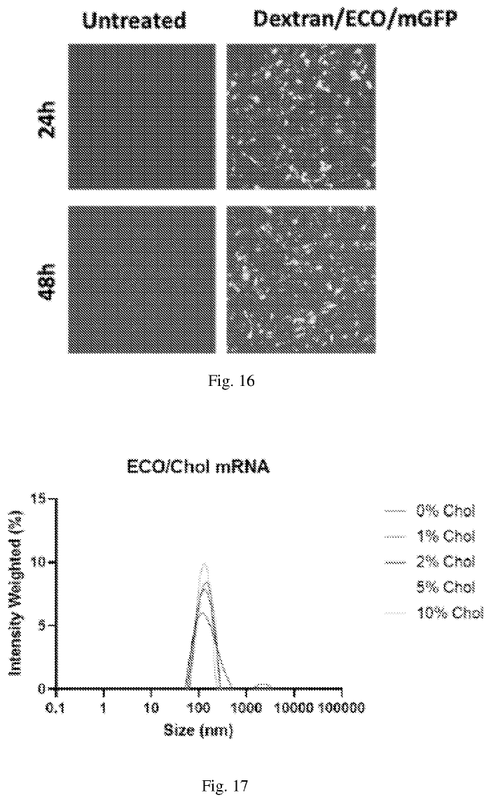

As demonstrated in FIG. 16, strong GFP expressions were observed in ARPE-19 cells 24 and 48 h after transfections using dextran-mal modified ECO/mRNA nanoparticles.

ECO/mRNA Nanoparticles can be Formulated with Helper Lipids Such as Cholesterol (Chol)

ECO/mRNA nanoparticles can be further formulated with helper lipids such as cholesterol (Chol). As previously described, ECO/mRNA nanoparticles were formulated at N/P ratio of 8 with mRNA that encodes EGFP. The final concentration of mRNA in the ECO/mRNA nanoparticle solution was 100 ng/μL. The ECO stock solution (50 mM in ethanol) and mRNA stock solution (0.5 mg/mL) at predetermined amounts based on the N/P ratio were mixed and vortexed for 15 min at room temperature. To formulated with cholesterol (Chol), Chol were added simultaneously with ECO at 1, 2, 5 and 10 mol % regarding the amount of ECO. The condensation of the mRNA with ECO and Chol into nanoparticles was verified by agarose gel electrophoresis prepared in a 0.7% agarose gel run at 100 V for 25 min. Size and zeta potential of the nanoparticles were determined using dynamic light scattering (DLS) with an Anton Paar Litesizer 500 (Anton Paar USA, Ashland, VA).

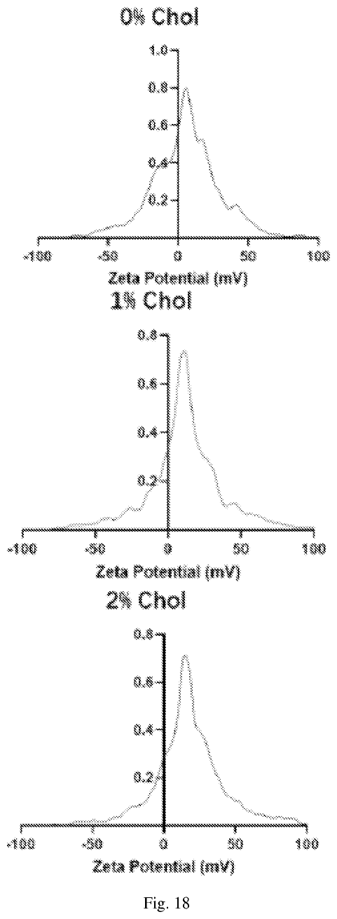

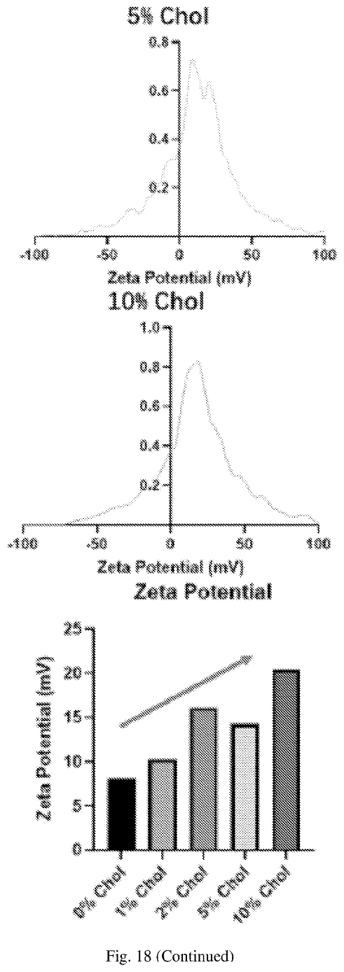

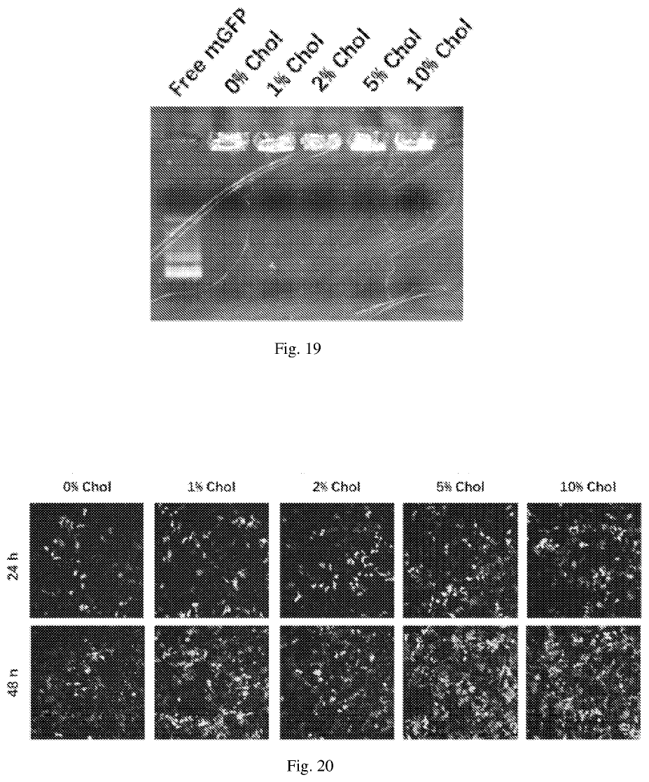

The results of ECO/mRNA nanoparticle formulated with Chol were shown in FIGS. 17, 18 and 19. ECO and mRNA could form stable nanoparticle with Chol at 1, 2, 5 and mol %. The nanoparticle structures became more compact after adding more Chol, indicated by the size distributions (FIG. 17). The zeta potential distributions shifted towards positive direction due to more compact structures with the increase of Chol amount (FIG. 18). The mRNA encapsulation was also confirmed by agarose gel electrophoresis FIG. 19. ECO could efficiently encapsulate mRNA both before and after Chol formulations across all the conditions.

ECO/mRNA Nanoparticles Formulated with Cholesterol (Chol) can Transfect Cells and Result in High Gene Expression

ECO/mRNA nanoparticles formulated with cholesterol (Chol) were tested in vitro transfection efficiency in HEK293T cells. ECO/mRNA nanoparticles were formulated at N/P ratio of 8 with mRNA that encodes EGFP. The final concentration of mRNA in the ECO/mRNA nanoparticle solution was 100 ng/μL. To formulated with cholesterol (Chol), Chol were added simultaneously with ECO at 1, 2, 5 and 10 mol % regarding the amount of ECO. HEK293T cells were seeded at 40,000 cells/well concentration on a 12-well plate. After the cells reached about 40-50% confluency, the nanoparticles were added to each well. GFP expression was evaluated using confocal microscopy at 24 and 48 h.

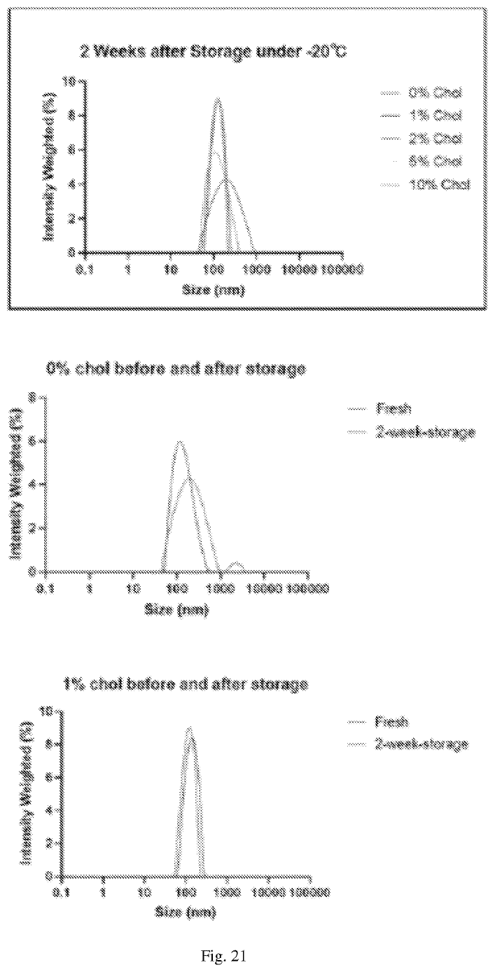

As demonstrated in FIG. 20, strong GFP expressions were observed in HEK293T cells both 24 and 48 h after transfections using ECO/mRNA nanoparticles formulated with Chol. There seemed to be an increase of GFP expression with the increase of Chol used in the ECO/mRNA formulations. Therefore, ECO/mRNA nanoparticles with Chol can result in stable nanoparticle formulations and excellent gene expressions.

ECO/mRNA Nanoparticles Formulated with Cholesterol (Chol) are Stable Under Storage Without Losing Transfection Efficiency

As previously described, ECO/mRNA nanoparticles can be further formulated with helper lipids such as cholesterol (Chol). To formulated with cholesterol (Chol), Chol were added simultaneously with ECO at 1, 2, 5 and 10 mol % regarding the amount of ECO. After the formulation, nanoparticle formulations were stored under −20° C. The stability of ECO/Chol/mRNA nanoparticle formulations was verified by checking the condensation of the mRNA with ECO and Chol by agarose gel electrophoresis prepared in a 0.7% agarose gel run at 100 V for 25 min. Size and zeta potential of the nanoparticles were determined using dynamic light scattering (DLS) with an Anton Paar Litesizer 500 (Anton Paar USA, Ashland, VA).

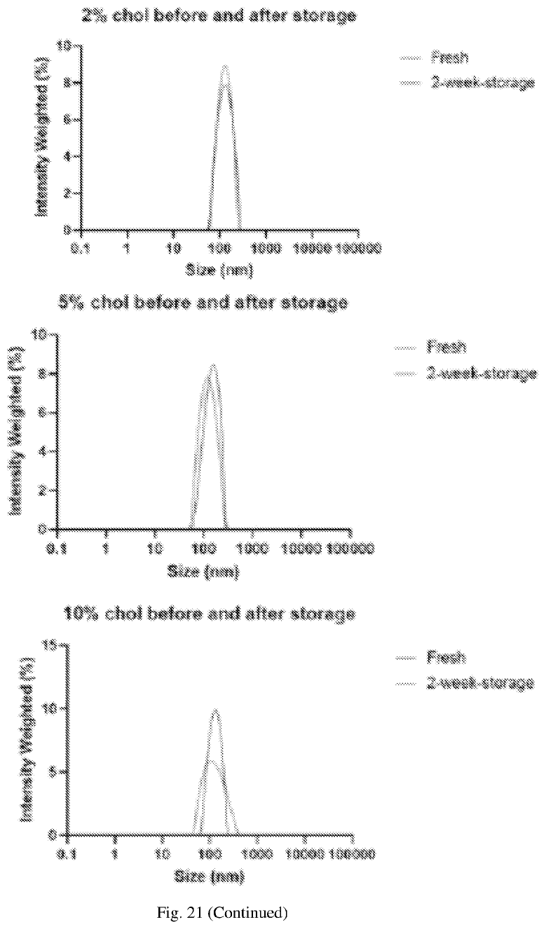

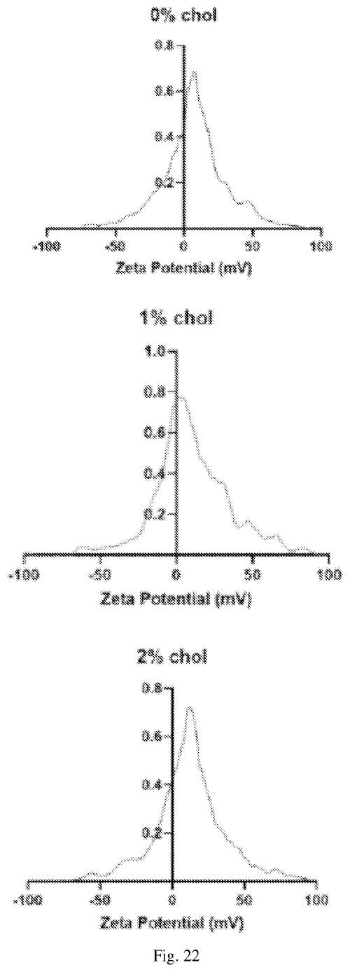

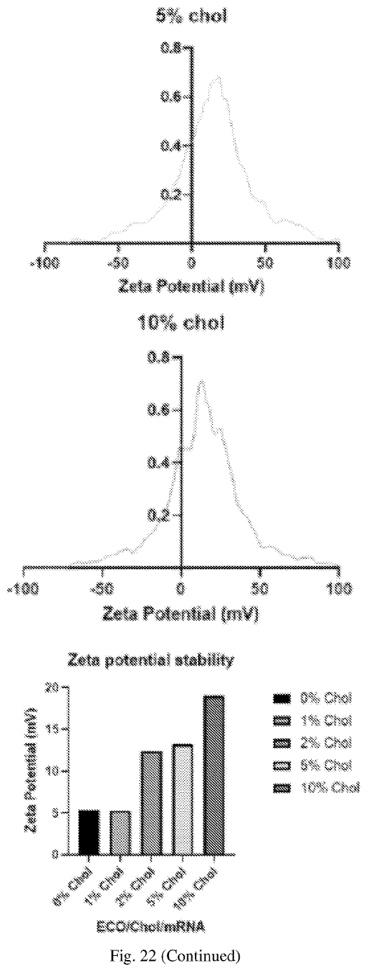

No significant change in the size distribution has been observed for ECO/Chol/mRNA nanoparticles after storage under −20° C. for 2 weeks. The distributions remained single peak with minimal aggregations (FIG. 21). Similarly, the zeta potential distribution and the increase of zeta potential with Chol content didn't change after storage if compared with freshly made nanoparticles (FIG. 18 and FIG. 22). Agarose electrophoresis also demonstrated that ECO/Chol/mRNA nanoparticles remain stable after storage (FIG. 23).

To check if storage affects the efficiency of ECO/Chol/mRNA nanoparticles, transfections were performed in HEK293T cells. HEK293T cells were seeded at 40,000 cells/well concentration on a 12-well plate. After the cells reached about 40~50% confluency, the nanoparticles after 2 weeks of storage under −20° C. were added to each well. GFP expression was evaluated using confocal microscopy at 24 and 48 h.

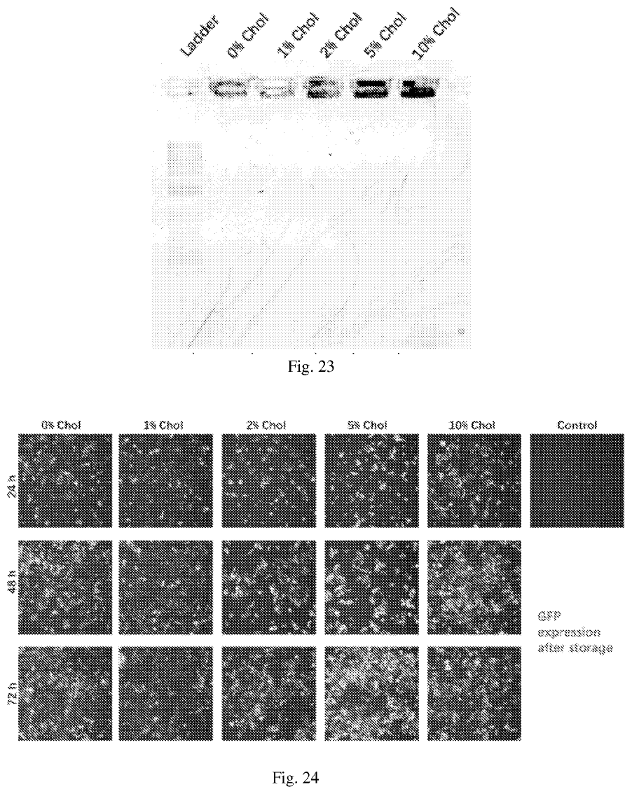

As demonstrated in FIG. 24, strong GFP expressions were observed in HEK293T cells 24, 48 and 72 h after transfections using ECO/Chol/mRNA nanoparticles after storage. No reduction in efficiency has been observed for ECO/Chol/mRNA nanoparticles after storage.

### End Functionalized Dextran (Dextran-Mono-Mal) can be Used for Surface Modifications of ECO/mRNA Nanoparticles

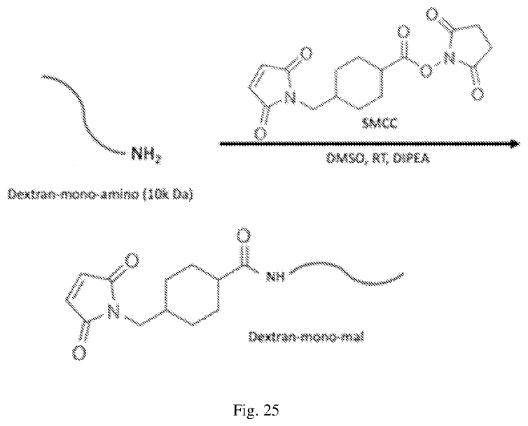

End functionalized dextran (dextran-mono-mal) was prepared via a chemical reaction between dextran-mono-amino and SMCC in DMSO (FIG. 25).

The end functionalized dextran with single maleimido group (dextran-mono-mal, 10 kDa) was used to modify ECO/mRNA nanoparticles. The process is similar to PEGylation of ECO/mRNA nanoparticles. As previously described, ECO/mRNA nanoparticles were formulated at N/P ratio of 8 with mRNA. The final concentration of mRNA in the ECO/mRNA nanoparticle solution was 100 ng/μL. The ECO stock solution (50 mM in ethanol) and mRNA stock solution (0.5 mg/mL) at predetermined amounts based on the N/P ratio were mixed and vortexed for 15 min at room temperature. After the ECO/mRNA nanoparticle was formed. Dextran-mono-mal (10 kDa) was added to the ECO/mRNA nanoparticle solution at 2.5 mol %, 3 mol %, 5 mol %, and 10 mol %, regarding the amount of ECO. Then the solution was shaken for another 15 minutes. The modified ECO/mRNA nanoparticle formulations can also be prepared through mixing dextran-mono-mal with ECO first for 15 min and then mixed with mRNA. Size and zeta potential of the nanoparticles were determined using dynamic light scattering (DLS) with an Anton Paar Litesizer 500 (Anton Paar USA, Ashland, VA).

TABLE 2
End functionalized dextran (dextran-mono-mal)
modified ECO/mRNA nanoparticle formulations.
DLS measurements of size, zeta potential and PDI

| | Dextran-mono-mal/ECO/mRNA | | |
|---|---|---|---|
| Dextran- | Size | Zeta | |
| mono-mal | (nm) | potential (mV) | PDI(&) |
| 2.50% | 152.47 | 52 | 24.9 |
| 3.00% | 110.55 | 21.5 | 16.3 |
| 5.00% | 128.3 | 35.1 | 22.5 |
| 10.00% | 210.5 | 51.2 | 24.2 |

ECO and mRNA could form stable nanoparticle with end functionalized dextran (dextran-mono-mal) at 2.5, 3, 5 and 10 mol % with sizes around 100-200 nm, positive zeta potentials and PDIs around 25% (Table 2).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A stabilized lipid nanoparticle comprising:
a) a plurality of pH sensitive protonatable or ionizable lipids having the structure of formula (I): 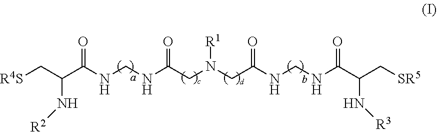
wherein R¹ is an alkyl group or an aromatic group, each of which is optionally substituted one or more hydroxyl group, ether group, or amino group;
R² and R³ are independently an aliphatic group or a hydrophobic group;
R⁴ and R⁵ are independently H, an alkyl group, an alkenyl group, an acyl group, or an aromatic group, or each R⁴ or R⁵ independently includes a polymer, or a polysaccharide, wherein each R⁴ or R⁵ is optionally substituted with a targeting group;
a, b, c, and d are independently an integer from 1 to 10; and pharmaceutically acceptable salts thereof;
b) at least one mRNA complexed with and/or encapsulated by the pH sensitive protonatable or ionizable lipids; and
c) a stabilizing amount of at least one stabilizing polymer, polysaccharide, or structural lipid that is conjugated to and/or complexed with the pH sensitive protonatable or ionizable lipids.

2: The stabilized lipid nanoparticle of claim 1, wherein R¹ comprises at least one of: 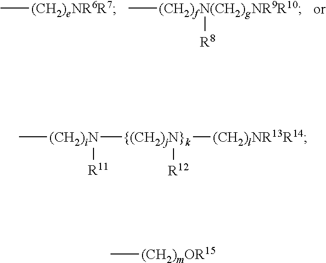
where R⁶, R⁷, R⁸, R⁹, R¹⁰, R¹¹, R¹², R¹³, R¹⁴, and R¹⁵ are independently hydrogen, an alkyl group, a hydrophobic group, a nitrogen containing substituent, or an oxygen containing substituent; and
e, f, g, i, j, k, l, and m are an integer from 1 to 10.

3: The stabilized lipid nanoparticle of claim 1, wherein a, b, c, and d are each 2.

4: The stabilized lipid nanoparticle of claim 1, wherein R¹ comprises at least one of CH₂CH₂NH₂, CH₂CH₂OH, CH₂CH₂OCH₂CH₂OH, CH₂CH₂OCH₂CH₂NH₂, CH₂CH₂NHCH₂CH₂NHCH₂CH₂NH, or CH₂CH₂NHCH₂CH₂CH₂CH₂NHCH₂CH₂CH₂NH.

5: The stabilized lipid nanoparticle of claim 1, wherein R² and R³ are each independently a saturated alkyl with long or branched chains, and or a fatty acid hydrophobic group derived from oleic acid or linoleic acid.

6. (canceled)

7: The stabilized lipid nanoparticle of claim 1, wherein at least one of R⁴ or R⁵ includes a polymer, or a polysaccharide, each optionally substituted with targeting group.

8: The stabilized lipid nanoparticle of claim 7, wherein the polymer or the polysaccharide of at least one of R⁴ or R⁵ is the stabilizing polymer or the stabilizing polysaccharide.

9: The stabilized lipid nanoparticle of claim 7, wherein the polymer comprises polyethylene glycol (PEG).

10: The stabilized lipid nanoparticle of claim 9, wherein the PEG has an average molecular weight of about 1,000 Daltons to about 100,000 Daltons.

11: The stabilized lipid nanoparticle of claim 7, wherein the polysaccharide comprises dextran.

12: The stabilized lipid nanoparticle of claim 11, wherein the dextran has an average molecular weight of about 1,000 to about 50,000.

13: The stabilized lipid nanoparticle of claim 11, wherein the dextran includes at one least side chain functionalized with a maleimide linker that is conjugated to a thiol group of formula (I) and/or a terminal end functionalized with a maleimide linker that is conjugated to a thiol group of formula (I).

14: The stabilized lipid nanoparticle of claim 1, wherein for at least some of the pH sensitive protonatable or ionizable lipids, R⁴ and R⁵ are each H, and for other of the pH sensitive protonatable or ionizable lipids, at least one of R⁴ or R⁵ includes a polymer, or a polysaccharide, each optionally substituted with targeting group.

15: The stabilized lipid nanoparticle of claim 1, wherein the pH sensitive protonatable or ionizable lipids are selected from: 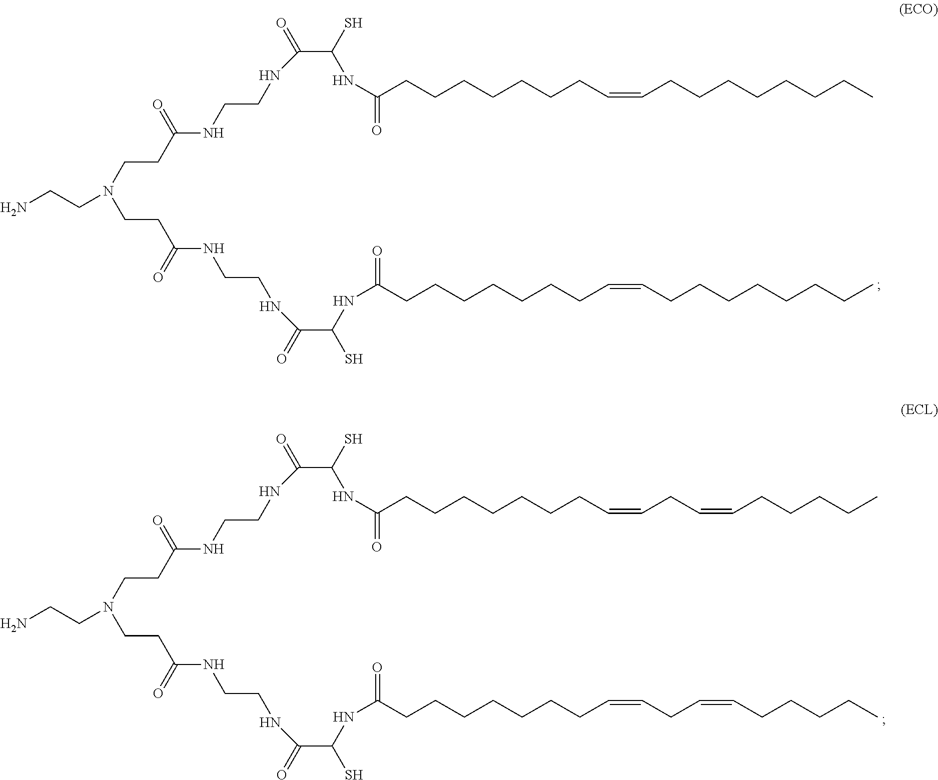 [SMILES: CCCCCCCC/C=C\CCCCCCCC(=O)NC(S)C(=O)NCCNC(=O)CCN(CCN)CCC(=O)NCCNC(=O)C(S)NC(=O)CCCCCCC/C=C\CCCCCCCC ; CCCCC/C=C\C/C=C\CCCCCCCC(=O)NC(S)C(=O)NCCNC(=O)CCN(CCN)CCC(=O)NCCNC(=O)C(S)NC(=O)CCCCCCC/C=C\C/C=C\CCCCC]
polyethylene glycol (PEG) modified lipids thereof, dextran modified lipids thereof, or combinations thereof.

16: The stabilized lipid nanoparticle of claim 15, wherein the plurality of pH sensitive protonatable or ionizable lipids include a plurality of ECO and/or ECLn lipids and a plurality of ECLn and/or ECO modified with PEG and/or dextran.

17: The stabilized lipid nanoparticle of claim 16, wherein the plurality of pH sensitive protonatable or ionizable lipids include about 1 mol % to about 30 mol % of ECLn and/or ECO modified with PEG and/or dextran.

18: The stabilized lipid nanoparticle of claim 1, wherein the targeting group is covalently attached to the polymer or polysaccharide.

19: The stabilized lipid nanoparticle of claim 1, wherein the stabilizing polysaccharide is hyaluronic acid that is complexed with the plurality of pH sensitive protonatable or ionizable lipids and mRNA.

20. (canceled)

21. (canceled)

22. (canceled)

23: The stabilized lipid nanoparticle of claim 1, wherein the structural lipid comprises cholesterol.

24. (canceled)

25: The stabilized lipid nanoparticle of claim 1, having an N/P ratio of about 2 to about 20.

26-32. (canceled)
